(19) United States
(12) Patent Application Publication
(10) Pub. No.: US 2007/0093968 A1
(43) Pub. Date: Apr. 26, 2007

(54) Selection of Genotyped transfusion donors by cross-matching genotyped recipients

(76) Inventors: Yi Zhang, Hillsborough, NJ (US); Michael Seul, Fanwood, NJ (US)

Correspondence Address: ERIC P. MIRABEZ, BIOARRY SOLUTIONS LLC, 35 TECHNOLOGY DRIVE, WARREN, NJ, 07059, US

(21) Appl. No.: 11/585,068

(22) Filed: Oct. 23, 2006

Related U.S. Application Data
(60) Provisional application No. 60/729,637, filed on Oct. 24, 2005.

(51) Int. Cl. G01N 33/567; G06F 19/00

(52) U.S. Cl. 702/19; 435/7.21

(57) ABSTRACT

Disclosed are methods for establishing the compatibility between two blood types on the basis of cross-matching (under a designated rule of stringency) the minor blood group genotypes of recipient and prospective donors. To determine compatibility, the blood group genotypes are mapped to corresponding phenotypes according to the expression states associated with a set of underlying haplotypes, and compatibility is established by establishing the compatibility of blood types constructed as a combination of constituent phenotypes. The bit strings are matched, preferably using an algorithm expression. Where ambiguity in mapping genotypes to haplotypes exists, it can be reduced based on frequency of occurrence of the haplotypes in the sample population, or resolved by gametic phasing. Such reduction or resolution of ambiguity is particularly desirable where mismatches in the antigens expressed by the constituent haplotypes have greater clinical significance.

### RELATED APPLICATIONS

This application claims priority to U.S. Provisional Application No. 60/729,637, filed Oct. 24, 2005.

### FIELD OF THE INVENTION

The invention relates to cross-matching of minor blood group antigens.

### BACKGROUND

At present, in the U.S., the compatibility between donor and recipient blood types is determined in accordance with a type & screen paradigm by typing of phenotypes, and screening recipients for alloantibodies against other antigens, and—only if such antibodies are detected—identifying the antibody, or antibodies, in order to select donor blood lacking the corresponding antigen(s) (“antigen-negative blood”) (Hillyer, C. D. et al., supra). The standard serological testing methodologies include: direct agglutination, immediate spin test, as well as indirect antiglobulin test (referred to as “IAT”; see I. Dunsford et al., Techniques in Blood Grouping, 2nd ed. Oliver and Boyd, Edinburgh (1967)). The IAT detects antibodies in the recipient's plasma that recognize antigens expressed on a donor's erythrocytes and thus can elicit a transfusion reaction. In fact, a cross-matching guideline on the basis of recipient and donor ABO/RhD phenotypes—in the form of a sequence of antibody screening, blood group checking, and delivery control (ABCD, see, e.g., J. Georgsen, et al., Transfusion service of the county of Funen. Organisational and economic aspects of restructuring. Ugeskrift for Laeger, 159, 1758-1762 (1997))—has been in routine use in the US, the UK, Sweden and Australia, where it has greatly expedited the process of identifying and issuing matched donor units while increasing the turnover of inventories and reducing routine labor. Computerized matching of donor and recipient which is used when the antibody screen is negative relies on the accuracy of the serological tests designed to determine the compatibility of recipient and donor blood for the major antigens, i.e., AB and RhD.

The selection of donor units that are known to be compatible for only the major antigens and/or known to be negative only to the specific antibodies implies a substantial risk of inducing alloimmune response related to the incompatibility between other blood group antigens, some of which are highly immunogenic and the presence of multiple antigenic factors may compound the adverse effect to a clinically significant level. Reducing the risk of allo-immunization thus remains an important clinical concern. This is so especially for poly-transfused patients, e.g., individuals suffering from sickle cell disease or hemophilia as well as patients with certain chronic diseases including cancer and diabetes. Each new allo-immunization increases the risk of patient morbidity. In addition, current practice can introduce delays in treatment and thus exacerbate emergency situations and more generally create significant additional expense in patient care.

The identification of antibodies and the provision of antigen-negative blood form the current approach to ensuring safe blood transfusion by seeking to minimize the risk of adverse transfusion reactions, triggered when antibodies circulating in the patient's blood stream encounter antigens displayed on a donor's erythrocytes. Reactions may vary in severity ranging from “none” to “severe” (Hillyer, C. D. et al., Blood blanking and transfusion medicine: basic principles and practice, Elsevier Science Health Science 2002, pp. 17). For instance, critical antigens in the ABO or Rh blood groups, if mismatched, can induce a severe adverse reaction, whereas antigen N, if mismatched, does not. The degree of severity also varies depending upon whether the subject is an adult or a newborn child. For example, an offending antigen S may cause only a mild adverse reaction in an adult but can cause severe hemolytic disease of the newborn. Although such qualitative descriptors are useful, a quantitative determination of compatibility of a prospective donor and a recipient would be more reliable, permitting acceptance evaluation and donor search to be conducted in a more objective and systematic fashion.

To prevent the transfusion of incompatible blood and reduce the risk of allo-immunization, it would be preferable to routinely type not only the major antigens but Rh variants and principal minor blood group antigens. However, the extension of routine serological typing to all clinically relevant antigens is precluded by the lack of appropriate antisera and the complexity and limited reliability of labor-intensive serological typing protocols, particularly when encountering multiple alloantibodies or weakly expressed antigens. In view of the limitations of serological testing methodologies, most donor centers screen only a selected cohort of donors for an extended set of antigens and maintain only a limited inventory. Sensitivity is another concern for the accuracy of the results. Since data interpretation in serotyping is based on the reaction patterns reflecting the amount of proteins on erythrocyte surface, signals are correlated with the expression levels of antigens to be probed. For example, antibodies directed against minor group antigens such as Duffy and Kidd may react less strongly when encountering cells bearing antigens reflecting heterozygous expression than against those reflecting homozygous expression.

In contrast, the analysis of blood group genes at the DNA level provides a detailed picture of the allelic diversity that underlies phenotypic variability. As recently described (Hashmi et al., Transfusion, 45, 680-688 (2005)), available methodologies permit the simultaneous analysis of clinically significant single nucleotide polymorphisms within the genes encoding the Kell, Duffy, Kidd, MNS and other antigens; these methodologies also lend themselves to the analysis of the highly variable RhD and RhCE genes (G. Hashmi et al., “Typing of Rh Variants using Bead Arrays on Semiconductor Chips”, Abstract S64-040B, American Association of Blood Banks (AABB) Annual Meeting, October 2004, Transfusion Vol 45 No. 3S, September 2005 Supplement), Human Leukocyte Antigens, Human Platelet Antigens and others. The benefit of cross-matching on the basis of genotypes relating to the expression of transfusion antigens is to minimize or eliminate not only the risk of adverse immune reactions, but also the risk of immunizing recipients in the first place, and to enable the rapid selection of blood products for transfusion from a group of donors. Genetic cross-matching would eliminate the need for costly serological reagents and complex and labor-intensive serological typing protocols, as well as the need for repeat testing of recipients for antibodies to particular donor antigens.

In addition, genetic cross-matching helps in addressing clinical problems that cannot be addressed by serological techniques, such as the determination of antigens for which the available antibodies are only weakly reactive, the analysis of recently transfused patients, or the identification of fetuses at risk for hemolytic disease of the newborn. Comprehensive DNA typing tools are becoming more accessible and cost-effective. They typically target a wide range of transfusion-related DNA markers. One example is comprehensive DNA typing based on eMAP, and performed in a BeadChip™ format (see “eMAP Application” U.S. Ser. No. 10/271,602; filed Oct. 15, 2002, incorporated by reference; see also Hashmi G. et al, A flexible array format for large-scale, rapid blood group DNA-typing, Transfusion, 45, 680-688 (2005); the latter reference describes a panel of comprising a set of 18 single nucleotide polymorphisms to resolve 36 alleles of Duffy, Dombrock, Landsteiner-Wiener, Colton, Scianna, Diego, Kidd, Kell, Lutheran and MNS systems). Beyond blood typing, broad-spectrum DNA typing extends knowledge to other related genetic repertoires such as those expressing Human Platelet Antigens, Human Leukocyte Antigens and others and has the potential to replace current serological methods as the routine method of characterizing recipients and selecting donors.

Thus, it will be useful to establish practical methods permitting the selection of compatible donors for a given recipient on the basis of transfusion antigen genotyping and to provide a quantitative risk assessment in the event of ambiguity to guide the selection of potentially only partially compatible donors who, given the limited availability of a diverse donor population, still may be desirable, while providing methods to reduce or eliminate that ambiguity.

### SUMMARY OF THE INVENTION

Disclosed are methods, representations and algorithms for establishing the compatibility between two blood types on the basis of cross-matching the transfusion antigen genotypes (also blood group genotypes) of recipient and prospective donor(s), a process also referred to as genetic Cross-Matching (“gXM”). To determine compatibility, the blood group genotypes are mapped to corresponding phenotypes according to the expression states associated with a set of underlying allelic combinations, and compatibility is established by establishing the compatibility of blood types constructed from constituent phenotypes.

Accordingly, a method for the rapid computational evaluation of compatibility between two blood types, that of a recipient (R) and a candidate donor (D), under a selected cross-matching rule of preset stringency is disclosed. For example, compatibility can be established under an exact rule, such that donor and recipient express the same set of antigens; alternatively, compatibility can be established under a relaxed rules, for example, such that the set of transfusion antigens expressed by the donor forms a subset of those expressed by the recipient (i.e., donor does not express any antigens recipient does not express and, in that sense, has a restricted antigen repertoire). To permit an effective computational implementation, blood types are represented in the form of binary strings (also “codes”, in one of several representations including octal and hexadecimal) such that subsets of bits within the string reflect the presence (“1”) or absence (“0”) of antigens defining individual phenotypes within blood group systems contributing to the specification of the blood type. The cross-matching rule, in accordance with the invention, is transcribed into a logical expression which is implemented computationally as a fast Boolean string matching operation to determine the compatibility between the R and D strings. Compatibility relationships between first and second sets of blood types, for example those most commonly observed in a given population, are conveniently displayed in a compatibility matrix, with, e.g., an entry of “1” indicating compatibility, and an entry of “0” indicating incompatibility. A measure of partial compatibility also is provided in terms of a product of scores associated with individual mismatched bits within the R and D strings, each mismatch score is set to a value between 0 and 1 to reflect the clinical significance of a mismatch between corresponding antigens. Compatibility and partial compatibility matrices are provided herein for the 25 16-antigen blood types most commonly observed (or expected) in African Americans on the basis of reported serological phenotype frequencies involving the minor blood group systems Duffy, Kell, Kidd, MNS, Dombrock and others.

Also disclosed is an algorithm and implementation of genotype to blood type mapping and genetic cross-matching. The algorithm permits establishing compatibility between a candidate donor and a recipient of known transfusion antigen genotype by way of mapping genotypes to phenotypes. Preferably, genotypes comprise the combinations of normal (N) and variant (V) allele assignments at each of multiple polymorphic sites within genes controlling the expression of selected transfusion antigens. Disclosed is a set of polymorphic transfusion antigen markers permitting the determination of compatibility by direct comparison of genotypes defined over that set of markers. More generally, the mapping invokes the decomposition of genotypes into constituent point mutation sets, herein termed “haplotypes,” that are combined under established rules of inheritance to determine the state of expression of encoded antigens defining specific phenotypes. In the event of ambiguity in the phenotype assignment, which generally arises when genotypes contain multi-site heterozygous diploids of unknown gametic phase, the algorithm permits the evaluation of partial phenotype compatibilities, as described in the first part herein, and provides a quantitative assessment of the risk associated with pairing the donor with the recipient; in addition, the algorithm permits the reduction of ambiguity by applying statistical haplotype analysis or resolution of the ambiguity by applying methods of determining an unknown gametic phase (also “phasing”).

### BRIEF DESCRIPTION OF THE DRAWINGS AND TABLES

FIG. 1 is a diagram illustrating mapping of genotypes to phenotypes to blood types and cross-matching in blood types.

FIG. 2 shows Venn diagrams illustrating the relationships between sets of expressed antigens of recipient and donor under different cross-matching rules.

FIG. 3 is a flow chart for a process identifying compatible donor blood for a recipient on the basis of transfusion antigen genotyping.

FIG. 4 illustrates gametic phasing by analyzing elongation products displayed on color-encoded microparticles.

FIG. 5 compares haplotype-derived 16-antigen minor-group blood-type frequencies in a population of 80 (self-identified) African American donors with frequencies derived by random combination of published serologically determined antigen frequencies.

FIG. 6 illustrates in a scatter plot the correlation shown in FIG. 5.

FIG. 7 (Table 1) lists the severity of an adverse reaction to transfusion of blood containing mismatched antigens, and related compatibility (also “mismatch”, MM) scores.

FIG. 8 (Table 2) shows antigen expression states determined by application of rules of inheritance specifying allele dominance relationships.

FIG. 9 (Table 3) shows a “one-to-one” mapping of genotypes to antigen phenotypes.

FIG. 10 (Table 4) shows a “many-to-one” mapping of genotypes to antigen phenotypes for the example of the Dombrock blood group system.

FIG. 11 (Table 5) shows a “one-to-many” mapping of genotypes to antigen phenotypes for the example of the Duffy blood group system.

FIG. 12 (Table 6) is a partial listing of phenotypes compatible to a given recipient phenotype.

FIG. 13 (Table 7) shows haplotypes of the Dombrock blood group system and corresponding antigen states.

FIG. 14 (Table 8) illustrates genotype-based cross-matching for a genotype DOB/HY and a corresponding phenotype, Do(a−b+).

FIG. 15 (Table 9) is a summary of genotypes compatible to genotype DOB/HY.

FIG. 16 (Table 10) illustrates haplotype analysis by inspection of genotype frequencies.

FIG. 17A (Table 11) lists the ten most common haplotypes and their frequencies for African Americans.

FIG. 17B (Table 12) lists the ten most common genotypes and their frequencies for African Americans.

FIG. 18 (Table 13) compares the 20 most common 16-antigen minor-group blood types and their genotype-derived frequencies in a population of 80 (self-identified) African Americans with frequencies derived by random combination of published serologically determined antigen frequencies.

FIG. 19 (Table 14) compares haplotype-derived phenotype frequencies with published serologically determined antigen frequencies.

FIG. 20 (Table 15) is a compatibility matrix for the 25 most common 16-antigen minor-group blood types in African Americans.

FIG. 21 (Table 16) is a partial compatibility matrix (threshold=0.5) for the 25 most common 16-antigen minor-group blood types in African Americans.

FIG. 22 (Table 17) shows genotype cross-matching.

FIG. 23 (Table 18) is a compatibility matrix for the 25 most common 16-antigen minor-group genotypes in African Americans.

FIG. 24 (Table 19) illustrates selection of compatible donor genotypes for a patient of known genotype in an African American population.

FIG. 25 (Table 20) is a partial compatibility matrix for the 50 most common 16-antigen minor-group blood types estimated from 80 self-identified African American donors.

FIG. 26 (Table 21) illustrates DNA-analysis derived antigen typing of two Caucasian individuals and cross-matching prediction and practice in an actual tri-state donor pool.

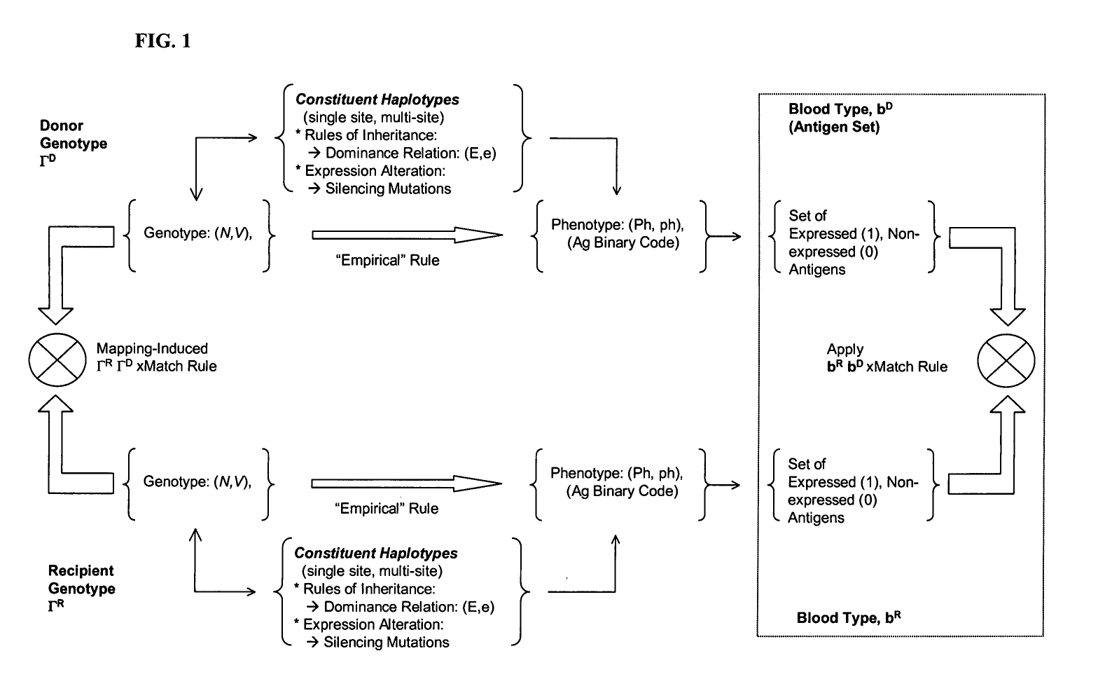

### DETAILED DESCRIPTION

I. Determination of Blood Type Compatibility. One prerequisite for the practical implementation of cross-matching is the need for establishing a mathematical representation of blood type and a compatibility scoring system to assess the effect of offending antigens which may induce adverse transfusion reactions at varying levels of severity. The effect of the alloantibodies, which may have been induced as a result of a previous transfusion including offending antigens (or antibodies acquired directly from the donor), also should be considered.

I.1 Representation of Blood Type (bT)—The combination of expressed (or weakly-expressed) antigens, summarized in a list, provides a convenient representation of a blood type in the form of a binary string, each bit indicating the presence (“1”) or absence (“0”) of a specific transfusion antigen. For example, if the known antigens are listed in the order: Fy^{a}, Fy^{b}, Lu^{a}, Lu^{b}, M, N, S, s, K, k, Jk^{a}, Jk^{b}, Do^{a}, Do^{b}, Hy, Jo(a), then the blood type code c010110101100111 represents a blood type: (Fy^{a}, Fy^{b}+, Lu^{a}−, Lu^{b}+, M+, N+, S−, s+, K−, k+, Jk^{a}+, Jk^{b}−, Do^{a}−, Do^{b}+, Hy+, Jo(a)+), characterized by the presence of antigens Fy^{b}, Lu^{b}, M, N, s, k, Jk^{a}, Do^{b}, Hy, and Jo(a) and the absence of antigens Fy^{a}, Lu^{a}, S, K, Jk^{b}, and Do^{a}. The code also can be expressed in hexadecimal form, i.e. c5F67. This definition of an individual's blood type also can include a record of alloantibodies to transfusion antigens other than that individual's own by listing the cognate antigens as “virtual” antigens. For example, if a donor has had a previous transfusion of only partially matched blood, all or some of the antigens displayed on transfused erythrocytes that are not expressed by the donor, the blood type string is augmented to contain a “0” entry for those “virtual” antigens. For example, if a sample from the previous transfusion donor were available for genotyping, antigens differing from the donor's could be included in the augmented recipient blood type. Specifically, if a donor, perhaps as a result of an earlier transfusion of only partially matched blood, is found to have formed an alloantibody against one of the mismatched antigens displayed on transfused erythrocytes, the blood type is augmented by an entry of “0” for the offending antigen. An entry of “1” for a virtual antigen could be used to indicate the absence of a specific alloantibody. This augmented representation ensures that compatibility scoring and cross-matching procedures, described below, remain correct for the entire augmented blood type.

I.2 Establishing Compatibility—The search for compatible donor(s), given a recipient of known blood type, requires the definition of a compatibility criterion, also referred to herein as a cross-matching rule.

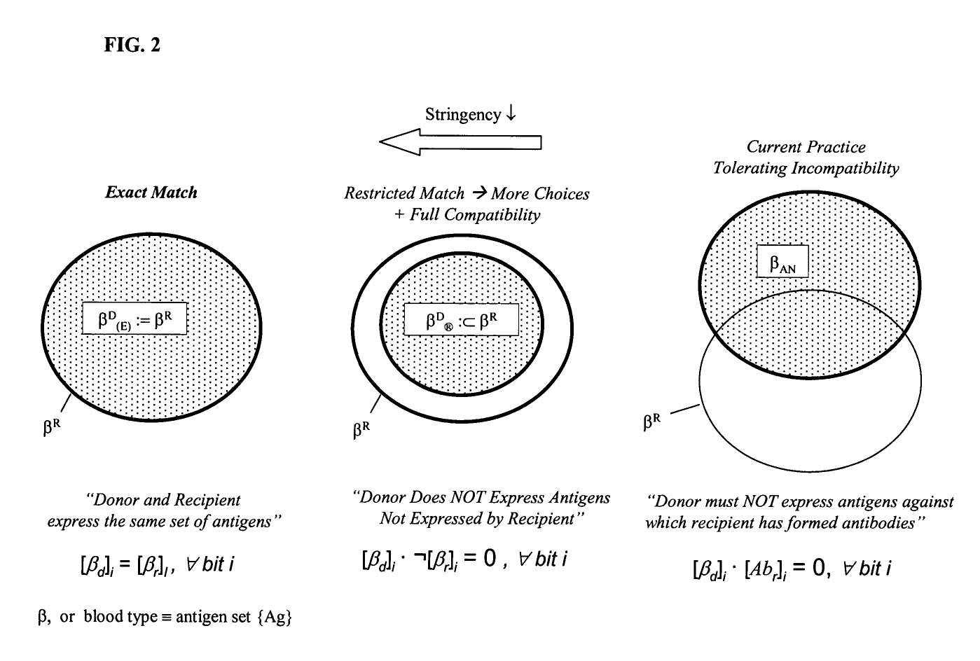

A first cross-matching rule, referred to herein as an Exact Cross-Matching Rule, states that a donor is compatible with a given recipient if donor and recipient express the same set of transfusion antigens selected for the comparison. A second cross-matching rule, referred to herein as a Relaxed Cross-Matching Rule, states that a donor is compatible with a given recipient if the donor does not express antigens that the recipient does not express—that is, the criterion enforces a restricted donor antigen repertoire. Under this rule, the set of selected antigens defining the donor blood type would be a subset of that defining the recipient blood type. Any blood type lacking antigens other than those displayed on the recipient's cells, in principle, should be compatible, because no reactive antibodies would be present in the recipient's serum to cause a transfusion reaction (so long as the recipient has not formed auto-antibodies, a rare condition that in any case will not be worsened by transfusion of donor blood as contemplated). The Relaxed Cross-Matching Rule would considerably expand the number of donors compatible with a given recipient compared to the Exact Cross-Matching, as illustrated in Example 3 and Example 5. A third rule, a variant of the Relaxed Cross-Matching Rule, states that a donor is considered partially compatible with a given recipient provided that the donor expresses only antigens that are “weakly” reactive with the recipient. A score is assigned reflecting the immunogenicity and corresponding clinical significance of those “offending” antigens, reflecting the speed and severity of an adverse response in the event of a mismatch. Current practice in transfusion is based on a cross-matching rules that selects compatible donor(s) based on the absence of antigens (antigen negatives), against which antibodies already have been formed in a recipient's blood. This rule unnecessarily permits the potential incompatibility between clinically significant antigens and the corresponding immunogenic reaction in recipient. FIG. 2 shows Venn diagrams illustrating the relationships between sets of expressed antigens of recipient and donor under different Cross-Matching Rules.

Under the Relaxed Cross-Matching Rule, the antigen repertoire of a prospective donor is restricted (compared to that of a donor selected under the Exact Cross-Matching Rule), because the donor repertoire of expressed antigens forms a subset of that of the given recipient. This restricted donor repertoire criterion may appear to limit the pool of prospective donors as it calls for donors having a smaller number of expressed antigens (or a larger number of “antigen negatives” in the conventional terminology). As a matter of fact, however, since the acceptable donor antigen subsets can be any combination of the recipient's antigens, the number of candidate donors who are compatible to a given recipient under Relaxed Cross-Matching is greater than that available under Exact Cross-Matching (see also Example 6).

For efficient implementation, cross-matching rules are transcribed into a logical expression, involving the strings, e.g., in binary, octal or hexadecimal form, representing the blood types of recipient and prospective donors. For the Relaxed Cross-Matching Rule, of particular significance to ensuring donor-recipient compatibility over an extended set of markers, the logical expression is {[β_{d}]ᵢ AND NOT[βᵣ]ᵢ}EQ 0, the index enumerating bits in the blood type strings. This expression yields a value of TRUE (“1”) when a bit in the donor blood type string is “1” AND the corresponding bit in the recipient's blood type string is “0”, indicating incompatibility.

Partial Compatibility—To establish a basis for the quantitative evaluation of partial compatibility, compatibility scores, ranging e.g. from 0 to 1, are assigned to antigens in the order of decreasing severity of adverse reactions in the event of a mismatch. That is, a non-immunogenic antigen is assigned a score of “1”, and a prohibitively immunogenic antigen is assigned a score of “0”. For example, ABO antigens, reflecting their clinical significance of causing “immediate; mild to severe” adverse transfusion reactions when mismatched, are assigned a score of “0”. In contrast, Lutheran antigens, reflecting their clinical significance of causing “delayed” adverse transfusion reactions when mismatched, are assigned a score of 0.75. The “look-up” Table 1 shows compatibility scores of some common transfusion antigens, if mismatched, based on their qualitative clinical reactivity ratings (Hillyer, C. D. et al., supra). Other definitions are possible, for example, in the form of a combination of the immunogenicity score with the frequency of occurrence of specific antigens and the severity of the elicited clinical reactions. An overall compatibility score is computed by multiplying compatibility scores of mismatched bits, which results in compounding the adverse effects when multiple immunoantigenetic entities are present. This assumption is consistent with the observation that, despite the fact that the immunization risk varies considerably for specific antigens, the additional antibody formation was shown to be independently associated with the number of transfusion episodes in a recent 20-year retrospective multicenter study (Schonewille et al, Transfusion, 46, 630-635 (2006)), provided that the current transfusion practice involves use of blood having antigen negatives specific to the identified antibodies, rather than blood that prevents immunization in the first place. Accordingly, denoting compatibility scores of individual antigens by {sᵢ}, elements in the blood type compatibility matrix are calculated in accordance with the expression:

$$e ( {β}_{d} , {β}_{r} ) = {\prod }_{i : {[ {β}_{d} ]}_{i} \cdot \overline{{[ {β}_{r} ]}_{i}} = 1} {s}_{i} , if \{ i \} \neq ϕ , \\ e ( {β}_{d} , {β}_{r} ) = 1 , if \{ i \} = ϕ ,$$

where [β_{d}] and [βᵣ] respectively denote the blood type codes of donor and recipient, and the index i refers to bits indicating the presence or absence of individual antigens in the blood type. The compatibility score, as a product of scores of all offending antigens, sᵢ, is thus bounded between 0 and 1. If set {i} is empty, there is no offending antigen; then, the result is 1 and donor's blood is considered fully compatible to the recipient; if the result is 0, donor's blood is considered incompatible. A fractional value of e expresses partial compatibility: the greater the value, the higher the degree of compatibility. In one embodiment, the partial compatibility score is thresholded, i.e., to set e(β_{d}, βᵣ):=0 if e(β_{d}, βᵣ)<eₜₕ, in order to exclude from consideration those blood types considered too risky for the purpose of transfusion.

Compatibility Matrix—Compatibility scores between first and second blood types observed or expected to be observed in a population can be compactly displayed in the form of a matrix. Each row, indexed by a specific first blood type, and rows ordered, for example, by decreasing frequency of occurrence of the selected blood types, contains a string composed of the scores indicating the degree of compatibility between the first blood type and second blood types in the selected set. According to the Exact Cross-Matching Rule, blood types are compatible with themselves—a situation also is referred to herein as an “e-Match”—indicated by diagonal matrix elements of “1”. Under the Relaxed Cross-Matching Rule, every first blood type may be compatible with several second blood types, and the corresponding (off-diagonal) elements of the matrix also will contain elements of “1”—a situation referred to herein as an “r-Match”, or an element showing the value obtained by evaluation of partial compatibility, as described—a situation also referred to herein as a “p-Match”. Matrix elements containing a value of zero indicate pairs of incompatible first and second blood types. In general, under the Relaxed Cross-Matching Rule, a first blood type representing a recipient blood type, may be compatible with several second blood types, representing candidate donor blood types, while the reverse does not hold: the matrix is not symmetric.

Assessing the Donor Pool—Ordinarily, transfusion donors may be disqualified if they have been previously the recipients of a blood transfusion that may have resulted in alloimmunization. In an emergency, however, such a donor may be acceptable under the current cross-matching rules as long as a compatibility score is calculated based on modified donor and recipient codes which at each “virtual” antigen position the recipient bit is copied to the donor bit and then set to “0”.

II: Determination of Transfusion Antigen Genotype Compatibility

II.1 Representation of Genotype—For present purposes, we define a transfusion genotype as a string of values giving the configuration (“allele”) of a target nucleic acid at specific variable sites (“loci”) within one or more genes of interest. Preferably, each designated site is interrogated with a pair of oligonucleotide probes of which one is designed to detect the normal (N) allele, the other to detect a specific variant (V) allele. Preferably elongation probes are used under conditions ensuring that polymerase-catalyzed probe elongation occurs for matched probes, that is those whose 3′ termini match corresponding marker alleles, but not for mismatched probes. The pattern of assay signal intensities representing the yield of individual probe elongation reactions in accordance with this eMAP™ format (see U.S. application Ser. No. 10/271,602, supra), is converted to a discrete reaction pattern—by application of preset thresholds—to ratios (or other combinations) of assay signal intensities associated with probes within a pair.

A genotype then is represented by a string, G={(NV)_{i,k}} where i enumerates the genes in the set of selected genes of interest, and k enumerates designated polymorphic sites within the i-th gene. N and V assume values representing an allelic state: in this disclosure, wild-type (or normal) and mutant (or variant) alleles preferably are denoted by the letters “A” and “B”, respectively. For example, at polymorphic site GYPB 143 T>C in the MNS system, “A” represents the normal allele, T, and “B” represents the variant allele, C. At loci having only two alleles, the biallelic combination, (NV), thus assumes values of AA, AB (or BA) and BB. Other letter(s) may be used to represent allelic state, for instance, a letter “D” stands for a deletion. In a preferred embodiment, the signal intensities associated with a pair of probes directed to the same marker, preferably corrected by removing non-specific (“background”) contributions, and one such intensity, I_{N}, associated with the probe detecting the normal allele, and the other such intensity, I_{V}, associated with the probe detecting the variant allele in the sample, are combined to form the discrimination parameter Δ=(I_{N}−I_{V})/(I_{N}+I_{V}), a quantity which varies between −1 and 1. For a given sample, a value of Δ below a preset lower threshold indicates homozygous variant, a value of Δ above a preset upper threshold indicates homozygous normal, and a value of Δ above the lower and below the upper threshold indicates a heterozygous configuration. A transfusion antigen genotype then also may be represented by a string, G={Δᵢₖ}, where, as before, i enumerates the genes in the set of selected genes of interest, and k enumerates designated polymorphic markers within the i-th gene. Accordingly, a transfusion antigen genotype is designated herein either in the representation AA, AB (or BA) and BB or, equivalently, in the representation 1, 0, −1. Genotypes represent the combination of two constituent strings, herein referred to as haplotypes, each representing a particular combination of allelic states at all marker sites—one allele per marker.

II.2 Selection of Markers—Testing for compatibility—for example identity, or near-identity, as described in greater detail below—of recipient and candidate donor is limited to a set of markers within relevant genes which, when expressed, encode certain human erythrocyte antigens (HEA) displayed on blood-borne cells against which the recipient either already has made (on the basis of earlier exposure) antibodies (“allo-antibodies”) or can make antibodies. A match, or near-match, between selected marker alleles identified in a recipient, and in candidate donors of transfused blood—the markers corresponding to polymorphic sites located in genes encoding blood group antigens and specifically including minor blood group antigens—generally will minimize the risk of recipient immunization and, in immunized recipients, the risk of alloantibody-mediated adverse transfusion reactions. That is, if the set of markers is selected to probe the relevant alleles associated with such reactions, then a comparison of marker alleles of recipient and donor can provide the basis for selecting compatible candidate donors. Sets of markers are disclosed in co-pending application Ser. No. 11/257,285 (see also: Example 2); these may be extended to include additional markers controlling expression, for example silencing mutations, and markers detecting deletions, insertions or recombinations.

To select donors in the general case, it would be desirable, in order to ensure the matching of all clinically relevant blood group antigens, to have a procedure for determining the compatibility of donors and recipients on the basis of comparing genotypes relating to the expression of clinically significant transfusion antigens.

II.3 Genotype-to-Blood Type Mapping—To implement genetic cross-matching in accordance with the invention, genotypes are mapped to blood types in a manner addressing ambiguity in the process (relating to the maxim that “the genotype is not the phenotype”); blood type compatibility is then evaluated using the methods disclosed in Part I. The determination of compatibility by genotype-to-phenotype mapping, in contrast to current practice invoking serological typing, affords superior reliability because both potentially “offending” entities contribute, that is, the transfusion-induced antibodies and “foreign” antigens on a donor's erythrocytes, as long as they are expressed, whether strongly or weakly. In many situations, the phenotype is directly and unambiguously identified by the genotype (Hashmi et al., supra). An issue addressed by the present invention is the quantitative assessment of risk relating to, and resolution of ambiguity arising from the degeneracy of mapping genotypes to phenotypes.

Given a genotype comprised of a designated set of alleles, the first step in blood type determination is to determine the state of expression of the individual transfusion antigens encoded by those alleles. For each marker, let (Ee) denote the dominance characteristic of alleles N and V in a genotype (NV), and let E and e assume one of three values—D (dominant gene), R (recessive gene), and N (non-expressed gene). The corresponding antigen expression states, (Ag^{N}Ag^{V}), reflecting the operative inheritance patterns, are then conveniently denoted by a pair of Boolean variables, (Xx), in which values of “1” (or “True”) and “0” (or “False”) respectively mark the presence and absence of an antigen, as described in Part I.

The value of (Xx) is determined by evaluating the following logic expressions:
X=(E EQ “D”) OR ((E EQ “R”) AND STATUS),
x=(e EQ “D”) OR ((e EQ “R”) AND STATUS),
   where
STATUS=(Ee NEQ “DR”) AND (Ee NEQ “RD”) AND (Ee NEQ “NN”).
   Here, OR, AND, EQ, and NEQ are logic operators that return Boolean values of “1” (“TRUE”) or “0” (“FALSE”), depending upon the validity of the corresponding “or”, “and”, “equal”, and “not equal” relationships, respectively.

“One-to-One” Mapping: SNP Markers (see also Example 2A)—Alleles in several important blood group systems comprise single nucleotide polymorphisms corresponding to single amino acid changes in the encoded antigens. In such cases, antigen expression states, (Xx), and thus phenotypes are readily and unambiguously evaluated from the expression above, as shown in Table 2 and Table 3: in the majority of cases of interest, alleles are co-dominant and antithetical antigens are expressed. For example, the single nucleotide polymorphism (SNP) JK 838 G>A in the Kidd system corresponds to a single amino acid substitution that changes the normal antigen, Jk^{a}, to the antithetical antigen, Jk^{b}.

“Many-to-One” Mapping (see also Example 2B)—In other instances, alleles comprise multiple variable loci. For example, as illustrated in Table 4, five variable loci within the Dombrock system at positions DO-793, DO-624, DO-378, DO-350 and DO-328, define a multiplicity of genotypes that, in some cases, represent more than a single combination of haplotypes. Remarkably, evaluation of the antigen expression states for individual haplotype combinations in accordance with known inheritance patterns (Reid, M. and Lomas-Francis, C., “The Blood Group Antigen Facts Book”, Academic Press, 2nd ed., 2004) shows that the different haplotype combinations (“diplotypes”) map to the same phenotype: for example, DOB/DOA and HA/SH both map to phenotype Do(a+b+), while multiple different genotypes map to each of the four (known) phenotypes. This situation is referred to herein as “many-to-one” (also “collapsed”) mapping.

The unambiguous mapping can be represented by the function:
ƒ_{gT→βT}:g_{r(d)}→β_{r(d)}.
If all antigens involved in defining a blood type are encoded by co-dominant alleles comprising single nucleotide polymorphisms corresponding to antithetical antigens, a special case of Cross-Matching—“g-match”, a fully compatible match—exists if recipient and donor have identical genotypes. For example, in this case of “one-to-one” mapping, identity of genotypes implies compatibility under the Exact Cross-Matching Rule.

“One-to-Many” Mapping: Ambiguity—More generally, the ambiguity implicit in 2-locus (or multi-locus) heterozygous genotypes with undetermined gametic phase admits of ambiguous phenotypes. For example (Table 5), a heterozygotic combination at the pair of loci FY-33 and FY125 in the Duffy system, depending on gametic phase, encodes either the antigen Fy^{a} or the antithetical antigen, Fy^{b}. That is, the normal allele, having a “G” at the site Duffy-Fy (FY125), encodes the antigen Fy^{a}, and the variable allele, having an “A” at that site, encodes the antithetical antigen, Fy^{b}, but expression is controlled by a separate marker, Duffy-GATA (FY-33): if Duffy-GATA (FY-33) is mutated, it disrupts transcription of the gene and silences expression of FYA/B. A 2-locus combination of heterozygous alleles, that is, (AB, AB) at {GATA, FY}, gives rise to ambiguity in phenotype prediction, for the haplotype combination can be either A-A/B-B, encoding Fy(a+b−) or A-B/B-A, encoding Fy(a−b+). Since the Duffy antigen, when mismatched in transfusion, can cause “mild to severe” transfusion reaction—as indicated by a partial compatibility score of e=0.375—the ambiguity in the genotype requires further elucidation. Methods of reducing or eliminating ambiguity by haplotype analysis are illustrated in Examples 3 and 4.

The ambiguous mapping can be described by the function:
ƒ_{gT→βT}:gᵣ→{βᵣᵥ}.

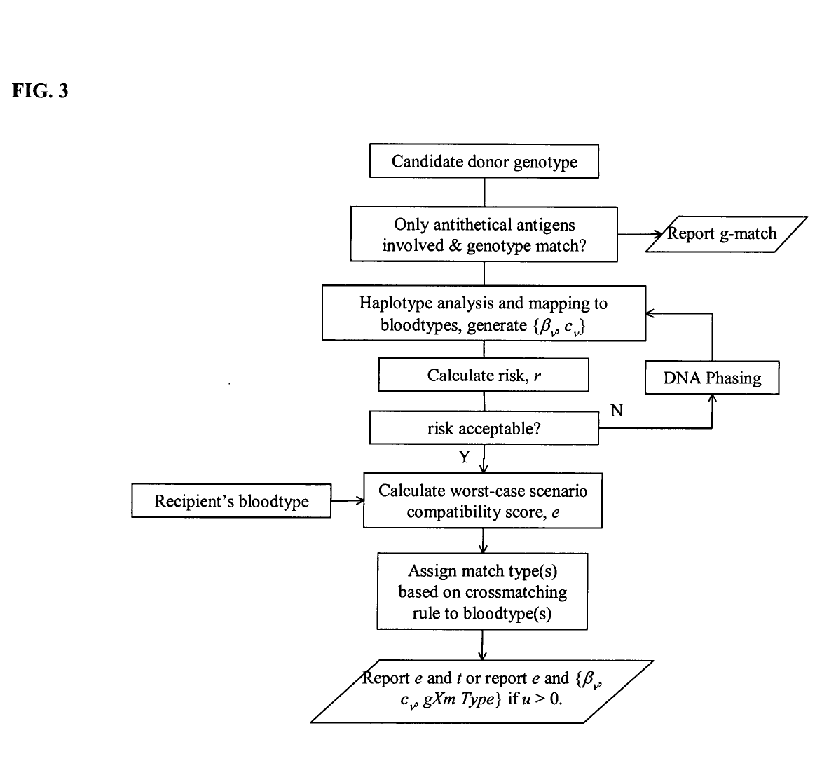

II.4 Assessment of Risk Associated with Mapping Ambiguity—The multiple potential (“phantom”) blood types produced by a “One-to-Many” mapping generally will differ in bits representing specific antigens—for example, the three phantom blood types c1001, c0001, and c000 differ in the first and last bits. The risk associated with mapping ambiguity and its potential clinical consequence thus manifests itself in the mismatched bits, and in the differing expression states of the corresponding potentially offending antigens. Especially in an emergency situation, it will be helpful to have a quantitative risk assessment relating to the ambiguity in a specific “One-to-Many” mapping, particularly when the determination is to be made for a recipient. A risk assessment is disclosed to provide a basis for deciding whether or not to accept the residual risk inherent in the ambiguity of specific phantom blood types and proceed, or seek additional clarification, in accordance with the procedure charted in FIG. 3.

One strategy is to proceed under the assumption of a “worst-case” scenario. That is, supposing the phantom blood types to be those of a recipient, compute the (partial) compatibility of all phantom blood types with all available candidate donors and adopt the lowest partial compatibility score as the basis for deciding whether or not to proceed. However, if the potentially offending antigens are clinically significant, the compatibility scores between the recipient's phantom blood types and the candidate donor blood types may differ widely, and the worst-case scenario may yield an overly conservative assessment. In addition, the frequency of occurrence of phantom blood types generally will not be identical. Thus, the worst-case scenario may relate to a phantom blood type with a low frequency. Prior to evaluating compatibility scores for all phantom blood types and available candidate donors, it is therefore advisable, in accordance with the strategy disclosed herein, to examine phantom blood types in greater detail. First, probabilities, {cᵥ}, are assigned to the potential (“phantom”) blood types that are consistent with the mapping in order to assess whether one or more of the phantom blood types may be rare. Next, viable phantom blood types are ranked in accordance with the {cᵥ} to define a risk threshold reflecting the likelihood of encountering a blood type with unacceptably low compatibility score. A risk score may be defined in the form of one of several possible combinations of the {cᵥ} and the compatibility scores.

Estimating Blood Type Frequencies—Blood type, defined herein as a combination of immunoantigenetic entities, typically contains more than 10 antigens, most of which are associated with highly polymorphic point mutations in genes. Estimating occurrence frequencies is critical for cross-matching donors and patients in a large scale, for example, in a blood center's database; nevertheless, an accurate estimation by direct counting is difficult, because the large number of combinations of those antigens dictates a sample of an impractically large size, in order for the results to have statistical significance. A desirable methodology described herein involves exploiting in subpopulations the linkage among the closely spaced point mutations along the same DNA stretch—alleles or haplotypes—and the statistical association among the those linked states on the different genes or chromosomes.

For alleles comprising multiple point mutations, especially when silencing mutation(s) are linked with the antigen determinant(s), haplotypes identified will be useful in deriving antigen expressions. For example, in a large-scale study mentioned in Example 9, GPB-int5 silencing mutation is confirmed as being always linked with a S-determining point mutation allele, GYPB^{S}, but never with mutant allele GYPB^{s}—in the other words, only haplotype, GPB-int5 “B”-GYPB^{S}, exists but not GPB-int5 “B”-GYPB^{s}. We will then have a greater confidence in assigning, for example, a typing of (GPB-int5 of AB and GPB of AB), S−s+ phenotype.

Haplotype analysis uses an expectation-maxinization (EM) algorithm to find linked states of point mutations along a short DNA stretch and to estimate their frequencies. A specific method commonly used in population genetics is gene counting, which is an EM algorithm for multinomial data (Weir B S. Genetic Data Analysis II: Methods for Discrete Population Genetic Data. Sunderland, M A: Sinauer Associates; 1996; Dempster A, Laird N, Rubin D. Maximum likelihood from incomplete data via the EM algorithm. Journal of Royal Statistical Society 1977; 39:1-38) in which haplotype frequencies (an underlying complete data set) can be estimated from genotype frequencies (a potentially incomplete data set determined in experiments) by an iterative method taking into account knowledge of the interdependence among parameters established (Lange K. Mathematical and Statistical Methods for Genetic Analysis. 2nd ed. New York: Springer; 2002.) Dipolotype frequencies are then calculated, following (Lange et al supra):

$${f ( H h )}_{d} = \{ \begin{matrix}2 \cdot f ( H ) \cdot f ( h ) & if H \neq h \\ {f ( H )}^{2} & if H = h .\end{matrix}$$

where H and h denote the two constituent haplotypes of a specific diplotype; the multiplication factor of 2 accounts for two equiprobable diplotypes composed of two haplotypes as they switch positions when inherited. The result forms a set of diplotype-frequency pairs—{dₖ, cₖ}. The occurrence frequency of a full set of point mutations, as one would inherit from one of the parents, herein termed “haplotype” in a broader sense, is then calculated as a product of occurrence frequencies of alleles/haplotypes on different genes, should they are tested non-associated. The probabilities of the “phantom” blood types, as estimated from haplotype analysis for recipient and/or donor, then may be written in the form:
ƒ_{gT→βT}:gᵣ→{βᵣᵥ,cᵥ^{r}}, and
ƒ_{gT→βT}:g_{d}→{β_{dμ},c_{μ}^{d}},

Phantom blood types with an estimated frequency below a preset threshold may be eliminated from further consideration without undue risk. Blood type frequencies can be then calculated as a product of occurrence frequencies of combinations of antigens in each blood group or gene, if they are tested non-associated, which in most cases is true. Otherwise, one needs to consider calculating the conditional probability of the occurrence of one arrangement that is conditional on another, which is located on a different gene or chromosome.

Following analysis of a small population sample, if a new genotype cannot be represented as a combination of established haplotypes, string matching may be attempted in search of new haplotypes that may form the given genotype in combination with any one of established haplotypes. This method in fact identified the two recently reported new haplotypes, Ha and Sh (Table 4) within the Dombrock system (Hashmi et al, supra). Frequencies of the new haplotypes are estimated by multiplying the frequencies of the constituent alleles, basically assuming a random combination, and the frequencies of the other haplotypes are appropriately renormalized. Then, the corresponding phantom blood types and their frequencies are recomputed in accordance with the expression given above. As the random donor pool accumulates more genotype cases, an EM calculation may be repeated in order to fine-tune the frequencies.

Computing a Risk Score—A quantitative measure of ambiguity may be obtained by comparing the phantom blood types to one another, preferably by adding up bits over corresponding positions in all strings. Any sum adding to a value other than either “0” or “N”, the number of phantom blood types, identifies a position at which at least one of the phantom blood types differs from the others, and in these positions, a checkbit is set. A clinically significant quantitative measure of the degree of ambiguity is then obtained by forming the product of compatibility scores (Table 1) associated with all the checkbit positions, in a manner analogous to the evaluation of partial compatibility described in Part I. A score, u, for the associated risk is determined by subtracting the product from unity:

$$\begin{matrix}u = 1 - {\prod }_{i \text{:} ∃ v \neq {v}^{′} , {[ β ]}_{i , v} \neq {[ β ]}_{i , {v}^{′}}} {s}_{i} , & if \{ i \} \neq Ø , \\ u = 0 , & if \{ i \} = Ø ,\end{matrix}$$

where the blood type, β, may be either βᵣ or β_{d}, respectively, for recipient or donor. If the product is close to unity—and the corresponding risk score, u, below a preset threshold—the difference among the phantom blood types is considered clinically insignificant. In such a case, it will be advisable to look for the “best case” scenario, that is, proceed with the donor producing the best compatibility score with any of the phantom blood types or by way of a linear combination:

$$ⅇ ( {g}_{d} , {g}_{r} ) = {\sum }_{μv} {c}_{μ}^{d} {c}_{v}^{r} ⅇ ( {β}_{d μ} , {β}_{r v} ) .$$

If the risk score is “high”, as indicated by a value of u exceeding a preset threshold, haplotype analysis (Examples 2 and 3) and optionally phasing (Example 4) may be performed at the discretion of the blood bank manager. In an emergency, should such additional analytical measures not be readily accessible in the available time, it may be advisable to reduce the degree of ambiguity by eliminating from consideration phantom blood types with estimated frequencies below a preset cutoff.

Partial Compatibility—Otherwise, partial compatibility scores are calculated for all viable phantom blood types. Should these have comparable estimated frequencies, and the ambiguity risk score is not high, a partial compatibility score may be determined as a frequency-weighted average. If, on the other hand, the ambiguity risk score is high, the partial compatibility score may be set in accordance with the “worst-case” assumption considered above by picking among all possible combinations of cross-matching between phantom blood types of a recipient and the most closely matched available donor blood type, the one with the lowest compatibility score:

$$ⅇ ( {g}_{d} , {β}_{r} ) = {min}_{μ , v \text{:} {c}_{μ}^{d} , {c}_{v}^{r} > {c}_{th}} ⌊ ⅇ ( {β}_{d μ} , {β}_{r v} ) ⌋ .$$

III. Compatible Donor Search and Cross-Matching Algorithm

With a binary (or equivalent) blood type representation defined, cross-matching rules of preset stringency established and transcribed into logical expressions, and a prescription for the assessment of risk associated with mapping ambiguity completed, a practical algorithm now is disclosed which incorporates these concepts and provides a method and implementation for the rapid selection of candidate donors for a given recipient on the basis of genotyping.

Given a pre-calculated compatibility matrix and a database of donor blood types derived by genotype-to-phenotype mapping, a fast-search algorithm can be implemented to identify candidate donors for a given recipient as follows.

First, construct a priority list in which potentially compatible blood types are enumerated. The list has three general sections: e (“exact”)-Match(es), r (“relaxed”)-Match(es), and p (“partial”)-Match(es)—in the order of descending priority. In e-Matches and r-Matches, the blood types with higher occurrence frequencies have higher priorities; in p-Matches, the blood types with higher compatibility scores have higher priorities. If multiple entries have the same compatibility score, more frequent types have higher priorities. Next, conduct a search of the priority list to find candidate donors following the priority order in the list; show all acceptably compatible candidate donors, keeping the priority order and attach the compatibility score for all candidate donors in the “partially compatible” category.

Implementation—Preferably, a computer program is used to implement the cross-matching procedure of the invention in the accordance with the pseudo-code outline below

| | |
|---|---|
| #define Dominant | 1 |

| | |
|---|---|
| #define Null | 0 |

| | |
|---|---|
| #define Recessive | −1 |

/* Subroutine for mapping genotypes to phenotypes at all markers for a given donor
geno-haplotype */
Geno2Pheno(DonorType, mapGeno2Pheno)
{

for (index = all markers in DonorType)
{

position=mapGeno2Pheno.find(DonorType.genotype);
DonorType.marker(index).phenotype=mapGeno2Pheno(position).second;

}

}
/* Subroutine for checking and setting expression states at all markers for a given donor
geno-haplotype */
checkExpressionState(DonorType)
{

for (index = all markers in DonorType)
{

/* find expression associated with each phenotype */
/* phenotype has the find-expression subroutine by looking up in

listPhenotypes*/

e1=DonorType.marker(index).phenotype1->

getExpression(listPhenotypes);

e2=DonorType.marker(index).phenotype2->

getExpression(listPhenotypes);

x1=(e1==Dominant)+(e1==Recessive)*((e1+e2)!=Null);
x2=(e2==Dominant)+(e2==Recessive)*((e1+e2)!=Null);
for (index2 = all haplotypes in DonorType)
{

if (associated haplotype suggests silencing at x1 or x2)

x1 or x2 =0;

}
/* Set the expression states on each allele on each marker */
DonorType(index).expression1=x1;
DonorType(index).expression1=x2;

}

}
/* Subroutine for mapping donor phenotypes to the blood type or a list of antigens */
Pheno2Blood(DonorType, mapPheno2Antigen)
{

for (index = all markers in DonorType)
{

for (x1, x2 that is true or expressed)
{

/* Find phenotype in the phenotype-to-antigen map */
position=

mapPheno2Antigen.find(DonorType.marker(index).phenotype);

/* Insert all found antigens to the existing list; repeated ones are

ignored */

DonorType.antigens.insert(mapPheno2Antigen.(position).

second);
}

}

}
/* Subroutine for establishing a list non-repeating blood types */
EstablishListBlood(DonorType, listBloods)
{

for (index = all elements in listDonorTypes)
{

if(listDonorTypes(index).antigens, the combination is not listed in the
listBlood)

listBlood.insert(listDonorTypes(index).antigens);

}

}
/* Subroutine for preprocessing */
Preprocess(listGenotypes, listPhenotypes, mapGeno2Pheno, listDonorTypes, listBloods)
{

/* Set the ID and name in a list of genotypes */
listGenotypes=setListGeno(fileParameters);
/* Set the ID, name, and expression state in a list of phenotypes */
listPhenotypes=setPhenoExpression(fileParameters);
/* Set genotype to phenotype map */
mapGeno2Pheno=setMapGeno2Pheno(fileParameters);
/* Set phenotype to antigen(s) map */
mapPheno2Antigen=setMapPheno2Antigen(fileParameters);
/* Map and associate the blood type to each donor geno-haplotype */
for (index=0 to listDonorTypes.size( ))
{

/* Same mapping procedure for all donors as in main( ) program for a

recipient */

Geno2Pheno(listDonorTypes(index).DonorType, mapGeno2Pheno);
checkExpressionState(listDonorTypes(index).DonorType);
Pheno2Blood(listDonorTypes(index).DonorType, mapPheno2Antigen);

}
EstablishListBlood(listDonorTypes, listBloods);

}
/* Genotype-based crossmatching */
main( )
{

/* Input all parameters, and map the donor genotypes to the blood type, */
/* and list all blood types*/
Preprocess(listGenotypes, listPhenotypes, mapGeno2Pheno, listDonorTypes,

listBloods);

/* Read recipient genotype from the request and map to blood type */
/* For each donor, genotype, phenotypes, expression states, and blood type and

code

are within “recipientType” data structure */

input(recipientGenotype);
input(ruleState);
recipientType.genotype=recipientGenotype;
/* Map genotype to phenotypes */
Geno2Pheno(recipientType, mapGeno2Pheno);
/* Check expression state alteration by haplotypes */
checkExpressionState(recipientType);
/* Map phenotypes to blood type and generate blood type code, which is a binary

string

itself or in hexadecimal form, with relative positions of bits following a preset
order of antigens */

Pheno2Blood(recipientType, mapPheno2Antigen);
[βᵣ]=recipientType.bTypeCode;
If (ruleState=EXACT)

for (index = listDonorTypes.size( ))
{

if(recipientType.bTypeCode==listDonorTypes(index).bTypeCode

print(listDonorType(index));, * Print out the result*/

}

else if (ruleState=RELAXED)

for (index = all listDonorTypes.size( ))
{

[β_{d}]=listDonorTypes(index).bTypeCode;
/* Check compatibility according to compatibility expression
matrix_element = ([β_{d}]&˜ [βᵣ]==0);
if(matrix_element!=0)

print(listDonorType(index)); /* Print out the result*/

}

else /* if ruleState = PARTIAL */

for (index = all listDonorTypes.size( ))
{

[β_{d}]=listDonorTypes(index).bTypeCode;
/* Check compatibility according to compatibility expression
/* 1. Calculate the code of offending antigens */
res = [β_{d}]&˜ [βᵣ];
/* 2. Calculate compatibility matrix element */
comp = 1.0;
for (i=0; i<bTypeLength; i++)

| | |
|---|---|
| if (res&(1<<i)) | /* If ith lowest bit is non-zero */ |

| | |
|---|---|
| comp*=s[i]; | /* multiply all s’ of offending |

antigens */

matrix_element = comp;
/* If non-zero element, print out the donor type and compatibility

value */

if(matrix_element!=0)

print(listDonorType(index), matrix_element);

}

}

### EXAMPLE 1

### Exact and Relaxed Cross-Matching Rules

Consider a blood type defined as a combination of phenotypes (Fy(a−b+), Lu(a−b+), M+N+S−s+, K−k+, Jk(a+b−), Do(a−b+)). According to one reference (Reid, M. & Lomas-Francis, C., supra) and analysis by random combination, this phenotype occurs with an approximate frequency of 1.5% in African Americans. Table 6 shows compatible full-phenotypes according to exact- and relaxed-matching rules. Under the Exact Cross-Matching Rule, a donor will have a full-phenotype identical to that of the recipient's. Under Relaxed Cross-Matching Rule, one would expect a null phenotype, Fy(a−b−), to be compatible with a recipient bearing the phenotype Fy(a−b+), since an erythrocyte having neither Fy^{a} nor Fy^{b} would display no potentially offending Duffy antigen to the recipient's immune system. The same reasoning applies to other markers. Thus, for instance, the combination—(Fy(a−b+), Lu(a−b+), M+N+S−s+, K−k+, Jk(a+b−), Do(a−b+)) would be considered a compatible type under the Relaxed Cross-Matching Rule under which a total of 54 phenotypes, corresponding to approximately 12.5% of available candidate donors, would be compatible, a proportion substantially exceeding that available under the Exact Cross-Matching Rule. Hence the name: Relaxed Cross-Matching Rule.

### EXAMPLE 2

### Genotype-to-Phenotype Mapping and Genotype Compatibility

This example illustrates the mapping of genotypes to phenotypes, and the combination of phenotypes into a blood type, followed by the application of cross-matching rules to phenotypes in order to derive sets of compatible genotypes. Genotypes, defined over a specific selection of 18 polymorphic loci relating to 26 phenotypes in Duffy, Lutheran, MNS, Kell, Kidd, Dombrock, Scianna, Diego, Colton, and Landsteiner-Wiener blood group systems, were identified using a panel of allele-specific probe pairs for 496 blood donors, stratified into several groups, as reported in Hashmi et al (supra).

2A—Direct Transcription by Visual Inspection—The single nucleotide polymorphisms defining alleles in the selected panel, all but those in Dombrock and Duffy blood group systems, have a one-to-one genotype-to-phenotype mapping, permitting the combination of corresponding antigens to be “read off” from the genotypes. For example, at Colton, the genotypes AA, AB, BB respectively corresponds to the antigen states (Co^{a}+, Co^{b}−), (Co^{a}+, Co^{b}+), (Co^{a}−, Co^{b}+). When A (“normal”) and B (“variant”) alleles are co-dominant, the cross-matching rules applying to genotypes are as follows: for exact cross-matching, all three types are only compatible to themselves and for relaxed cross-matching, AA and BB are compatible to themselves and all three types are compatible to AB.

2B—Multilocus Alleles and Statistical Haplotype Analysis: Dombrock—For the Dombrock blood group system, alleles, defined in terms of five polymorphic loci: DO-793, DO-624, DO-378, DO-350 and DO-323, encode four (out of five known) antigens, i.e., Do^{a}, Do^{b}, Holley (Hy), and Joseph (Jo(a)). When phenotypes are determined by multi-locus alleles, visual inspection generally will be insufficient to construct the mapping. To proceed, haplotypes must be constructed to account for the observed genotypes, and by applying established rules of inheritance, phenotypes are identified. Statistical haplotype analysis provides a well-established methodology for identification of the most likely set of haplotypes to account for the observed distribution of genotypes.

Testing the published typing results for the entire set of 18 loci (relating to 36 pairs of alleles) for Hardy Weinberg equilibrium yielded P-values greater than 0.1, indicating alleles to be equilibrated in the population, and further indicating that sampling and typing errors were negligible. An Expectation-Maximization (EM) algorithm (see Dempster A P, et al., “Maximum Likelihood from Incomplete Data via the EM Algorithm”, J. R. Stat. Soc. B 1997: 39: 1-38), in a publicly available implementation, HAPLORE (Zhang K, et al., “HAPLORE: a program for haplotype reconstruction in general pedigrees without recombination”, Bioinformatics 2005: 21:90-103), was used to estimate haplotype frequencies to account for the reported genotype frequencies. As an input to HAPLORE, a pedigree file was constructed from the set of encountered allele types, A or B at each polymorphic locus, which were each assigned an internal ID, i.e., 1 or 2. The convergence criterion relating to the incremental relative improvement of haplotype frequency estimates in successive EM iterations was set to 10⁻⁸, and the frequency threshold to retain a haplotype was set to 10⁻⁶. The algorithm not only identified the six haplotypes previously reported (Hashmi et al, supra), but also provided corresponding estimated frequencies. With reference to the literature for the relevant rules of inheritance, all antigen states were readily constructed from these haplotypes and phenotype frequencies estimated (not shown).

Table 7 lists the results, and Table 8 summarizes the mapping of Dombrock genotypes to their corresponding phenotypes and antigen states. For example, genotype DOB/DOB maps to phenotype Do(a−^{b}+) and then to an antigen state of (Do^{a}−, Do^{b}+, Hy+, Jo(a)+), with antigen code 0111. Remarkably, as previously observed (Hashmi et al, supra), while, in several cases, multiple distinct haplotype combinations were found to produce the same genotype, all these combinations, along with other genotypes, were found to map to the same blood type, permitting, in this instance, to infer from the identity of recipient and donor genotypes the compatibility of Dombrock phenotypes. More systematically, a compatibility matrix associates recipient antigen codes with their compatible donor antigen codes using a selected cross-matching rule. For example, the compatibility matrix connects the donor code 0111 to recipient codes, 0111 and 1111.

Reverse Mapping and Genotype Compatibility—Given a phenotype compatibility matrix, the mapping in Table 8 yields compatible sets of donor genotypes. For example, given a genotype of DOB/HY, the corresponding phenotype is first identified as Do(a−b+), with antigen code 0111. As illustrated in the table, to identify a compatible genotype, a search is initiated to connect code 0111 (indicated by a dotted circle) to two compatible donor antigen codes, 0111 and 0101. The first code, 0111, corresponds to a compatibility element along the diagonal of the matrix, indicating an exact cross-match. Five compatible genotypes are found: DOB/DOB, DOB/HY, DOB/SH, HY/SH and SH/SH; the full set of compatible genotypes is listed in Table 9. The second code, 0101, corresponds to an off-diagonal element in the compatibility matrix, indicating a relaxed cross-match. Only one compatible genotype, HY/HY, is found. Table 4 summarizes all compatible genotypes, showing genotypes compatible under the Relaxed Cross-Matching Rule in italics. If a phenotype for the recipient is already known, one simply skips the mapping and starts from the antigen code.

### EXAMPLE 3

### Reducing Ambiguity by Elimination: GATA-Duffy

Heterozygosity at two biallelic loci without resolution of the gametic phase, generally implies ambiguity. However, in certain situations, especially when the absence of Hardy Weinberg equilibrium suggests non-random sampling, it may be possible to resolve the ambiguity by inspection of the data. A case in point is the combination of FY-33, a silencing mutation in the GATA box of Duffy, and the marker at FY125, denoted FYA. /FYB. Table 10 shows genotype frequencies for the GATA mutation and FYA/FYB as observed in a set of 430 random donors of unspecified ethnic origin, in the aforementioned published data set (Hashmi et al., supra), Hardy-Weinberg Equilibrium testing (not shown here) suggests the donor population to be strongly stratified, precluding application of the EM algorithm. However, direct inspection provides the requisite insight. Thus, 2-locus biallelic combinations of {GATA, FY} yielding the observed genotypes are listed (middle panel in Table 10) along with observed frequencies (lower panel in Table 10). All elements of the table are readily assigned except for (AB, AB). Inspection of the observed genotypes along the row and column of haplotype B-A reveals that none of the corresponding combinations—(AB, AA), (BB, AA), and (BB, AB)—are observed. This strongly indicates the absence of haplotype B-A and the identification of the combination (A-A/B-B) to unambiguously account for genotype (AB, AB).

### EXAMPLE 4

### Resolution of Haplotype Ambiguity by DNA Phasing

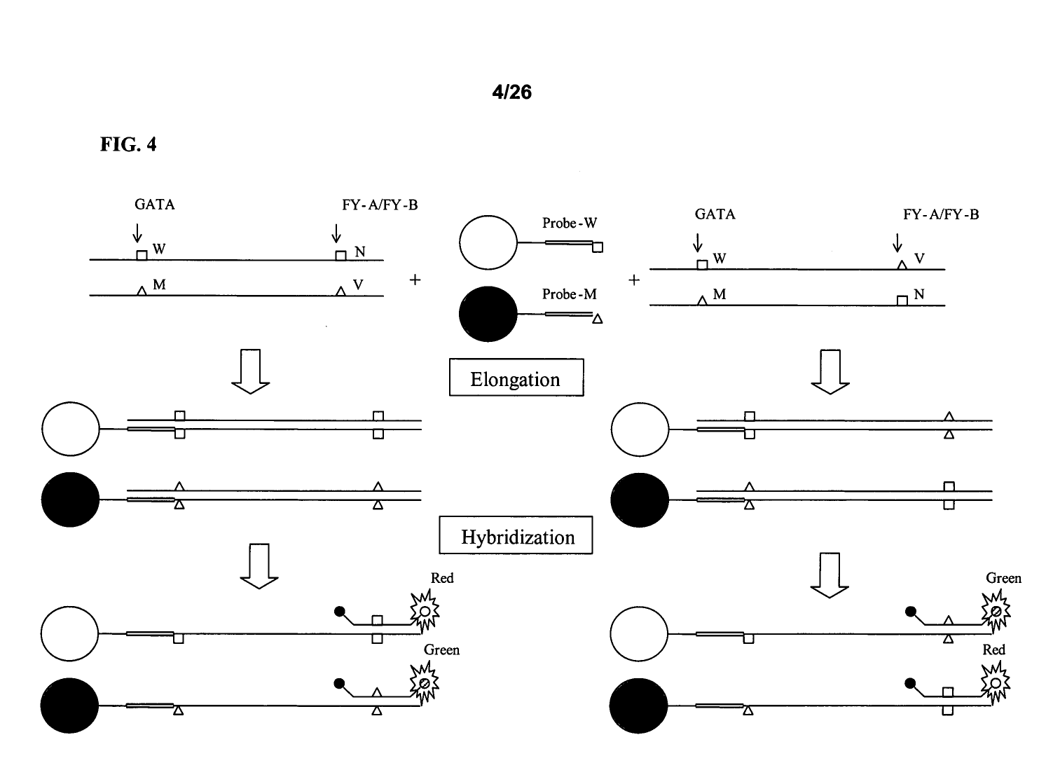

This example illustrates the use of phasing to resolve ambiguity arising from heterozygosity at two or more biallelic loci when neither application of statistical haplotype analysis nor direct visual inspection reduces ambiguity to an acceptable level, or eliminates it altogether. As shown in FIG. 4 for the GATA-Duffy configuration of the previous Example, phasing, invoking probe elongation, preferably in the BeadChip™ format (see U.S. application Ser. No. 11/257,285; U.S. application Ser. No. 10/271,602 (“eMAP”), both incorporated by reference) comprises the following four steps: (a) providing a pair of two degenerate probes on color-encoded beads, under conditions permitting the target to anneal to the probe so as to bring the 3′ termini of the two probes into alignment with a designated polymorphic site within the target; as illustrated for GATA-Duffy (FIG. 4), the 3′-terminus of one probe (probe-W) is designed to be complementary to the GATA wild-type allele and the 3-terminus of the other probe (probe-M) is designed to be complementary to the GATA mutated allele; (b) under appropriate conditions, allowing the targets (PCR amplicons) to hybridize and a DNA polymerase such as ThermoSequenase, which lacks 3′ to 5′ exonuclease activity, to attach and specifically elongate the probe whose 3′-terminus is complementary to the target, in this example at FY-33; (c) under stringent condition, separating DNA hybrids; (d) optionally, washing and removing target strands; and (e) analyzing the elongation product by hybridizing to a second variable site of interest within elongation product, in this example at FY125, two detection probes, one, probe-N is labeled, for example in red fluorescence color and directed to the normal allele, the other, probe-V, is labeled, for example in green fluorescence color and directed to the variant allele. The probes preferably are designed in the configuration of a molecular beacon or a looped probe (US application Ser. No. 10/032,657) in order to minimize the fluorescence background in solution. FIG. 4 illustrates the possible outcomes: if the bead displaying probe-W shows red color and the bead displaying probe-M shows green color, the haplotype is W-N/M-V; if, instead, the bead displaying probe-W shows green color and the bead displaying probe-M shows red color, the haplotype is W-V/M-N. The gametic phase of the two heterozygous biallelic haplotypes is thus resolved, and the ambiguity in the mapping of the observed genotype to a phenotype is eliminated.

### EXAMPLE 5

### Genotype-Derived Blood Types in African American Donor Population

This example presents an analysis of an unpublished data set of transfusion antigen genotypes in a small population of (self-identified) African American donors and confirms the validity of genotype-derived blood types from the standpoint of population genetics.

Blood samples were collected from 80 unrelated African American New York City donors, and DNA-typing was performed using a panel of 18 allele-specific probe pairs to identify alleles associated with 26 phenotypes in Duffy, Lutheran, MNS, Kell, Kidd, Dombrock, Scianna, Diego, Colton, and Landsteiner-Wiener blood group systems, and hemoglobin S, a hemoglobin mutation associated with sickle cell disease, as previously reported (Hashmi et al., supra). Since no variant alleles were observed in Scianna, Diego, Colton, Landsteiner-Wiener systems, and HbS, so they are considered by default matched in this exercise.

Haplotype Determination—Genotype data for all markers were first tested for Hardy-Weinberg equilibrium (HWE) by performing an exact test on the selected set of SNPs using the program PEDSTATS (Wigginton et al., Bioinformatics 2005 21(16): 3445-3447). Pedigree files were constructed to indicate individuals to be unrelated. Data files were constructed to include the marker names. The result showed equilibrium at all markers, with p values ranging from 0.04 to 1, with the exception of GPA, which encodes the M/N antigens in the MNS group, and showed a p value <0.005. The negligible overall deviation from HWE suggested that errors from sampling and genotyping were minimal. The sample size, 80, nevertheless was small relative to the over 300 different genotypes observed in the data set in Example 2, and the actual experimental counts are thus expected to be of limited reliability in estimating the frequencies of the genotype-derived blood types.

The first step in this analysis is to reconstruct underlying haplotypes and to estimate their frequencies by gene counting and expectation-maximization (“EM”) (Dempster et al, supra) in each blood group. The EM algorithm has been applied to population genetics to estimate haplotype frequencies (an underlying complete data set) from genotype frequencies (an incomplete experimentally determined data set) by an iterative method taking into account knowledge of interdependence among parameters established, in this case, by way of gene counting; an implementation of EM is provided in the program, HAPLORE, (see the reference in Example 2). As input, HAPLORE uses a pedigree file constructed from possible combinations of alleles, denoted, for example, by A for the normal (most prevalent) and B for a variant. The convergence criterion relating to the incremental relative improvement of haplotype frequency estimates in successive iterations was set to 10⁻⁸, and the frequency threshold to retain a haplotype was set to 10⁻⁶. Haplotypes and alleles among different genes were tested for association, which was found none. The ten most common point mutation sets, or broader-sense “haplotypes”, and genotypes, so established for African Americans, with their associated frequencies, are listed in Table 11 and Table 12, respectively.

Out of 2¹⁷ possible combinations, 44 haplotypes defined over the set {GATA, FY, FY-265, GPA, GPB, K, Jk, DO-323, DO-350, DO-378, DO-624, DO-793, Lu, SC, DI, CO, LW} were found to have significantly high frequencies. The most common haplotype, with a frequency of 23.2%, was found to be B-B-A˜A-B˜B-A˜A-A-B-B-B˜B˜A˜A˜A˜A, and the 10 most common haplotypes were found to account for 65% of all haplotypes identified in the test population. The swung dash represents statistical association among the SNPs that are located at different chromosomes. The most common genotype, with a frequency of 6%, was found to be (BB, BB, AA, AB, BB, BB, AA, AA, AA, BB, BB, BB, BB, AA, AA, AA, AA). The 10 most common genotypes account for 28% of all genotypes in the test population.

Remarkably, in all 44 identified haplotypes, the mutation at FY-33T>C (Duffy GATA) appears in conjunction with the variant allele FY125G>A, implying the silencing of the variant antigen, Fy^{b} (see also Example 3). That is, expectation maximization confirms the observation, previously reported on the basis of serological typing (Reid & Lomas-Francis, supra) that the 2-locus GATA-Duffy genotype (AB, AB) at {GATA, FY}, in African Americans, always has a diplotype (A-A, B-B), corresponding to phenotype Fy(a+b−). This observation explains why the serologically determined frequency of the encoded antigen, Fy^{b} of 23%, counting both Fy(a−^{b}+) and Fy(a+^{b}+) frequencies (Reid & Lomas-Francis, supra), is significantly lower than the observed allele frequency 91% for the variant FYA/FYB.

Mapping—The resolution of the GATA-Duffy ambiguity permits unambiguous genotype-to-phenotype mapping, shown in Tables 3 and 4; genotype (AB, AB) at {GATA, FY} now is assigned to antigen code 10 at {Fy^{a}, Fy^{b}}.

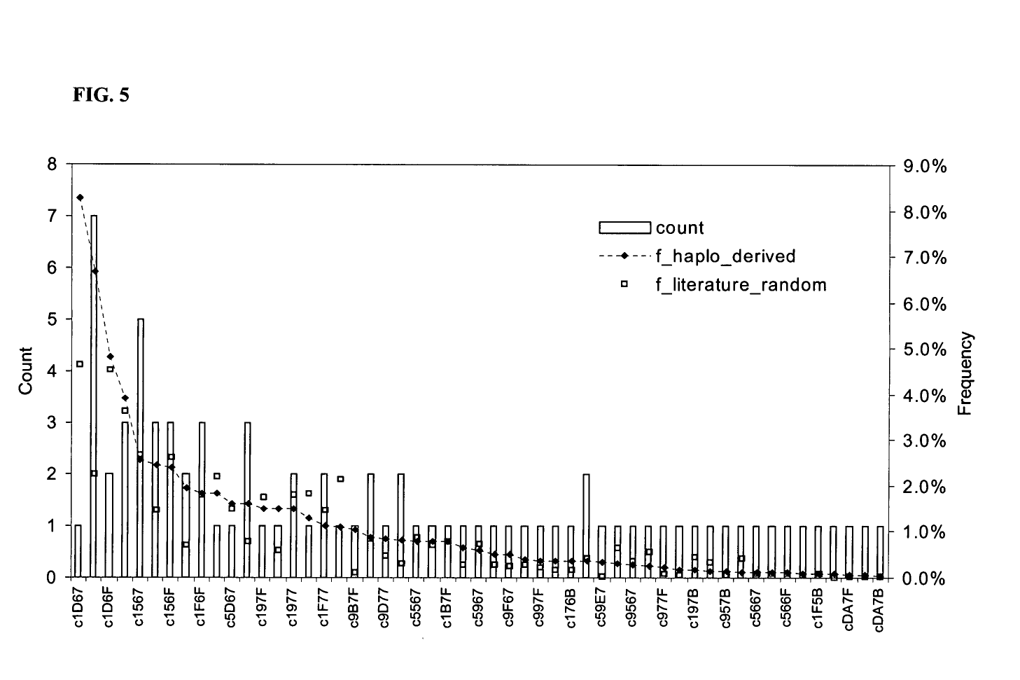

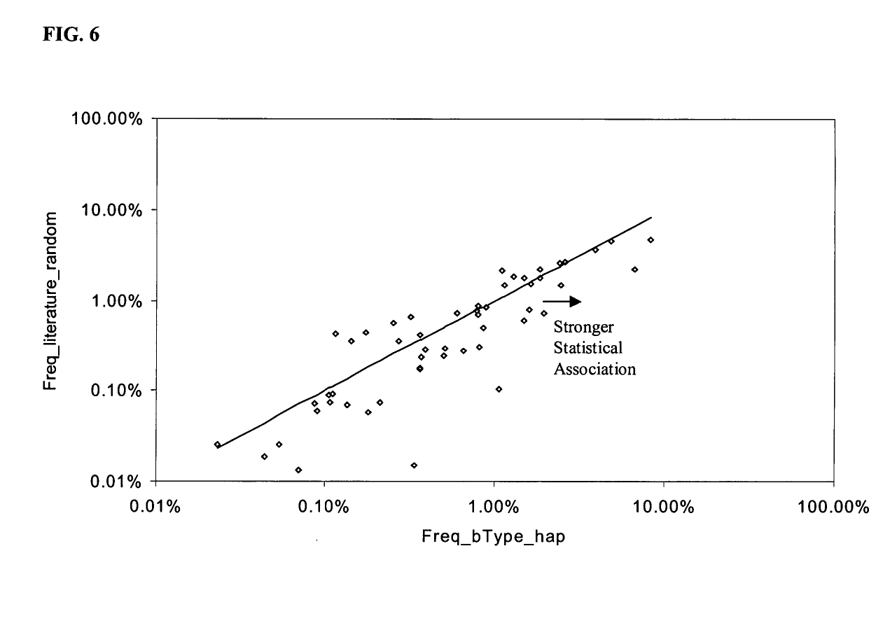

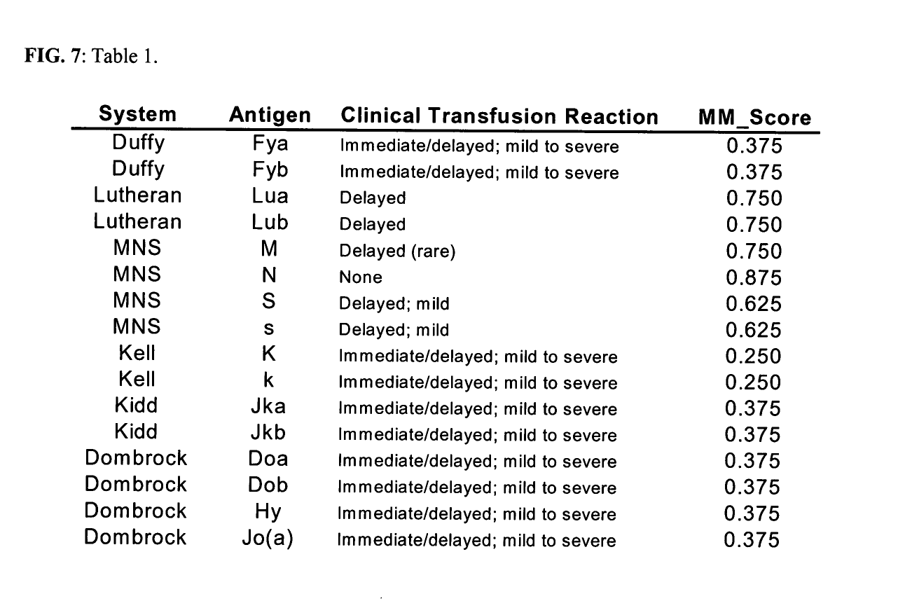

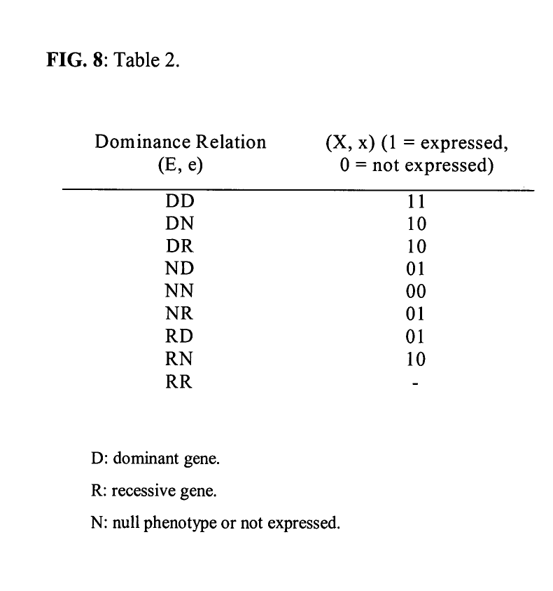

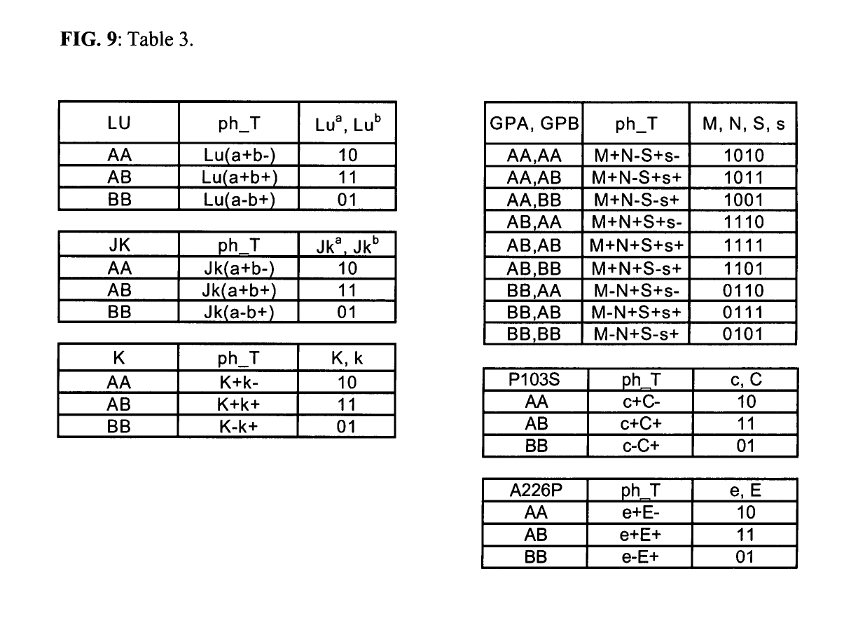

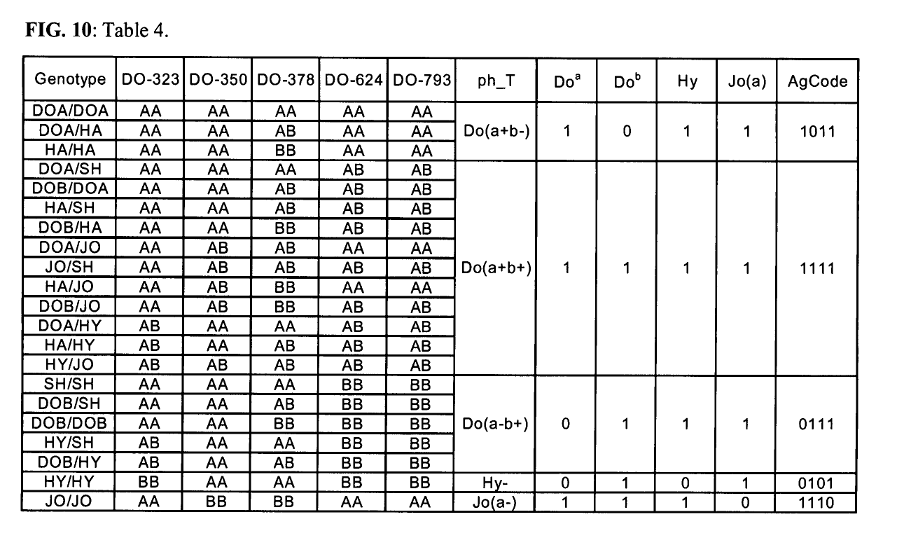

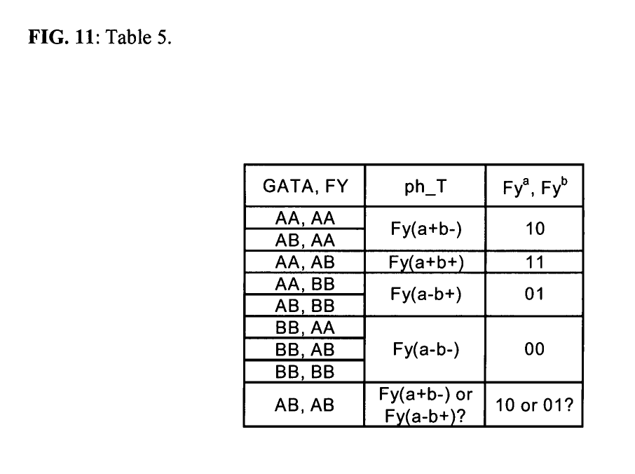

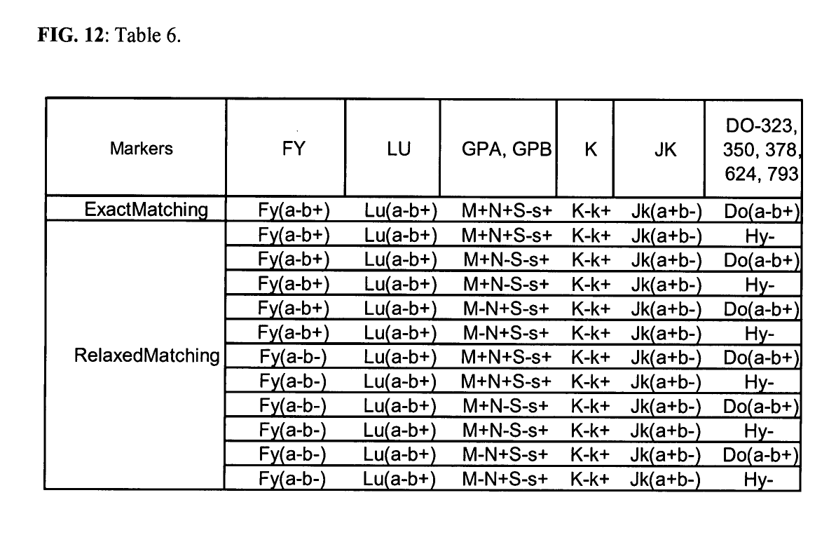

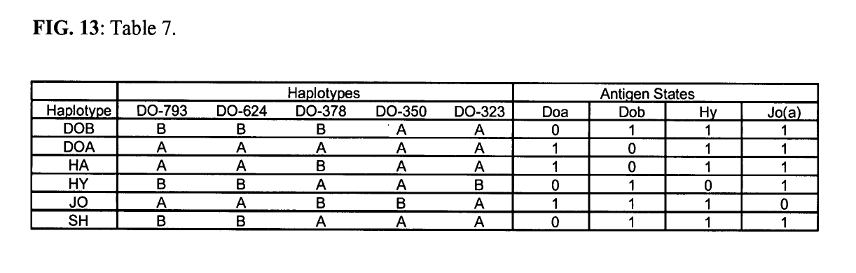

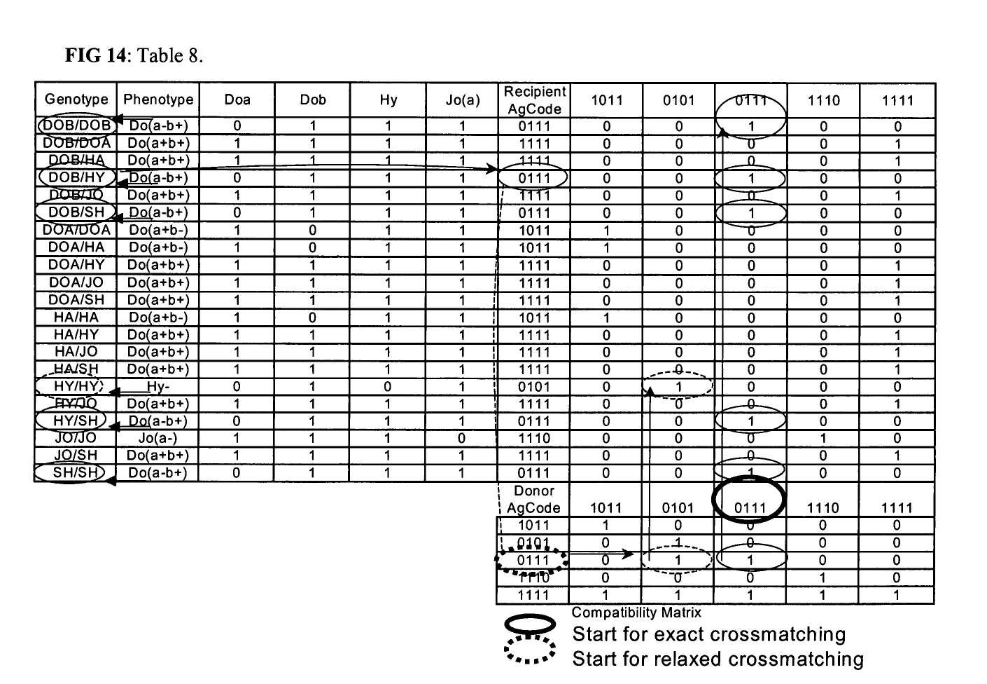

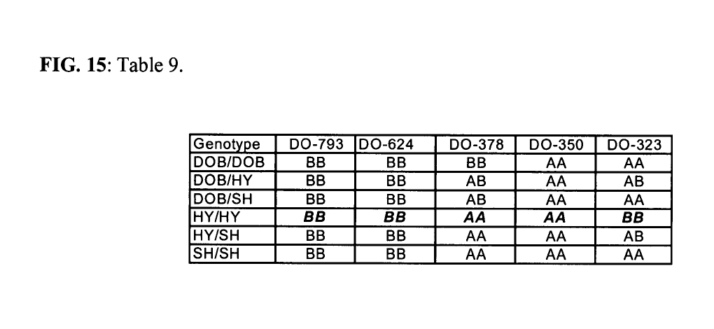

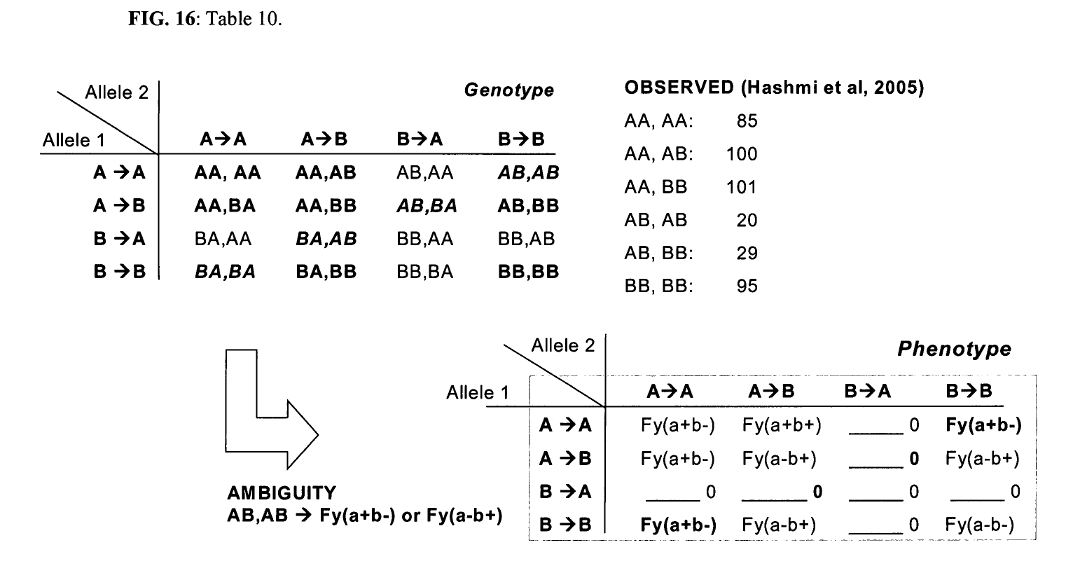

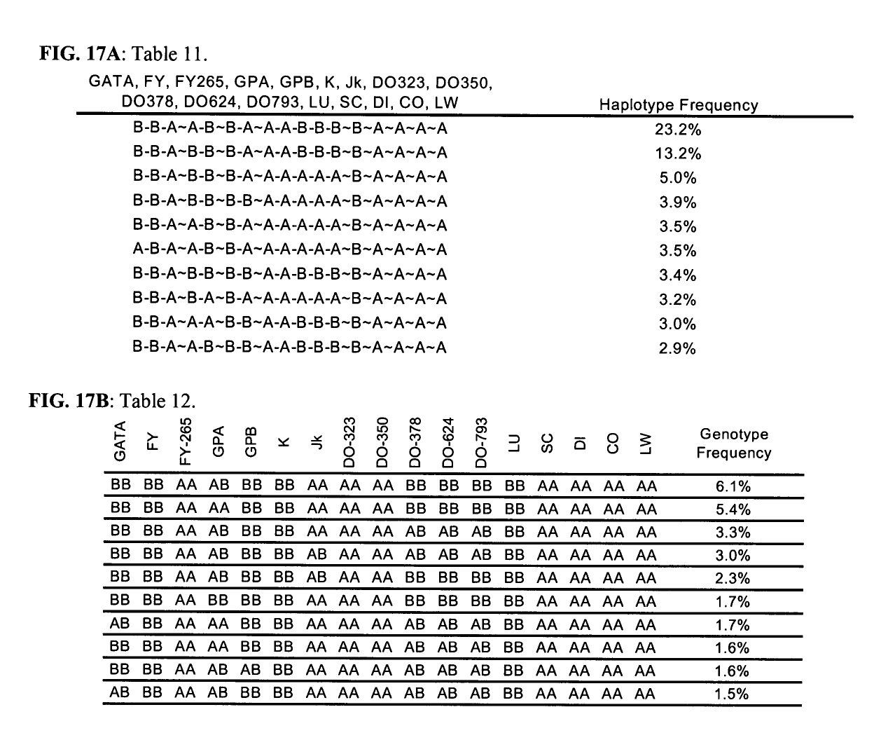

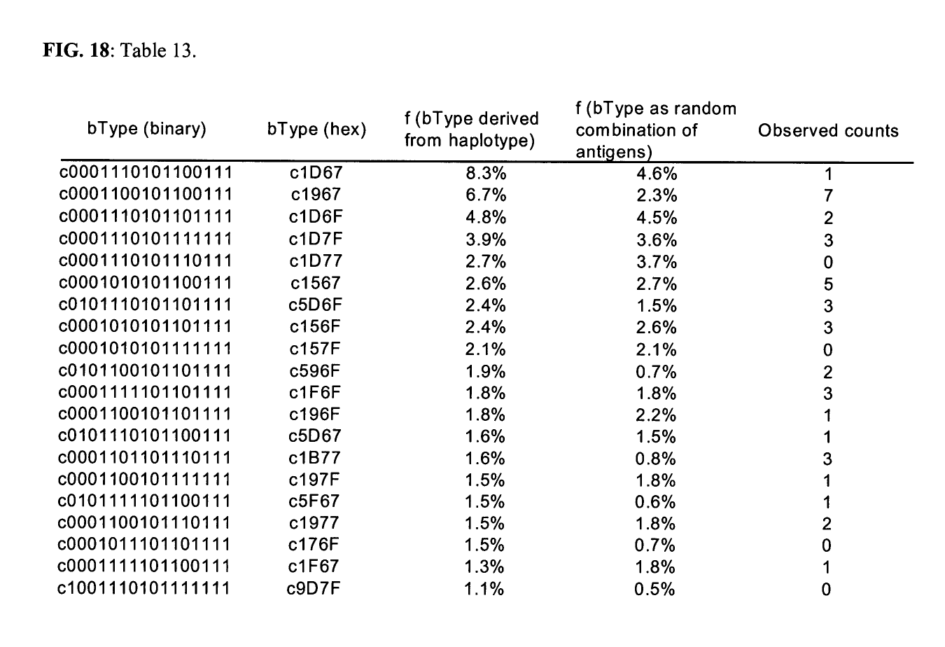

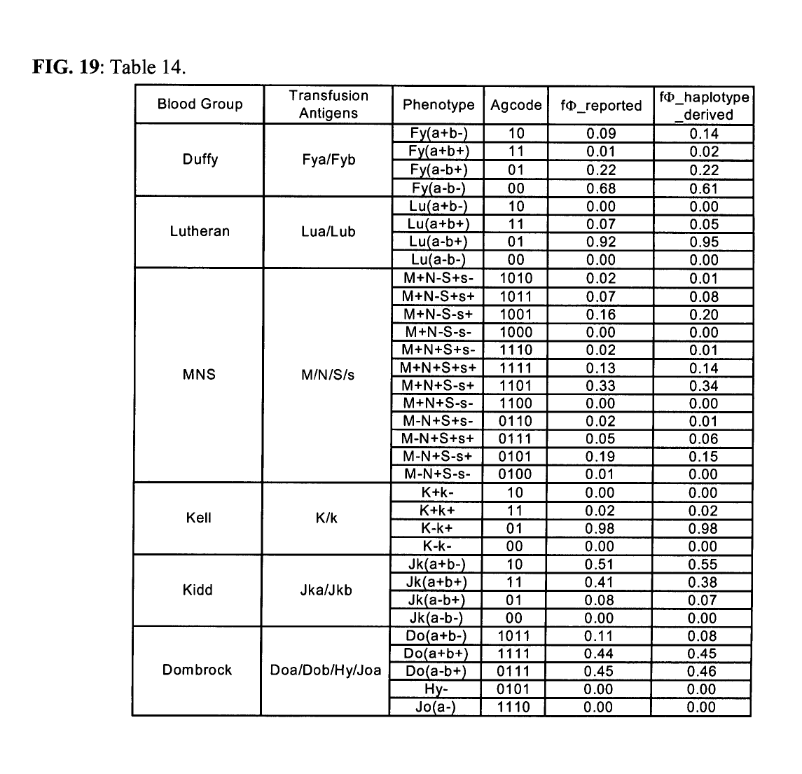

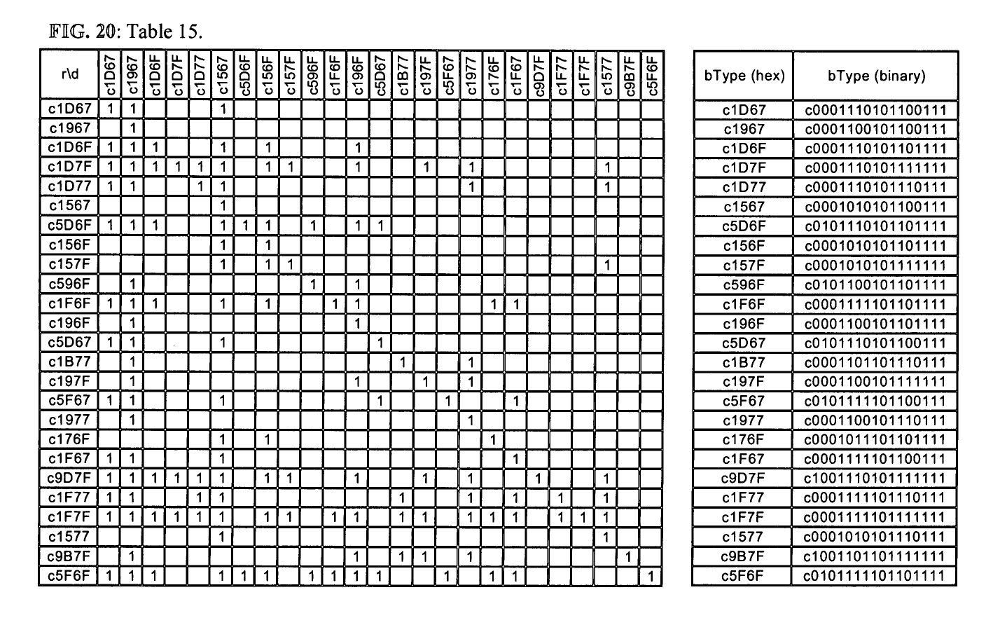

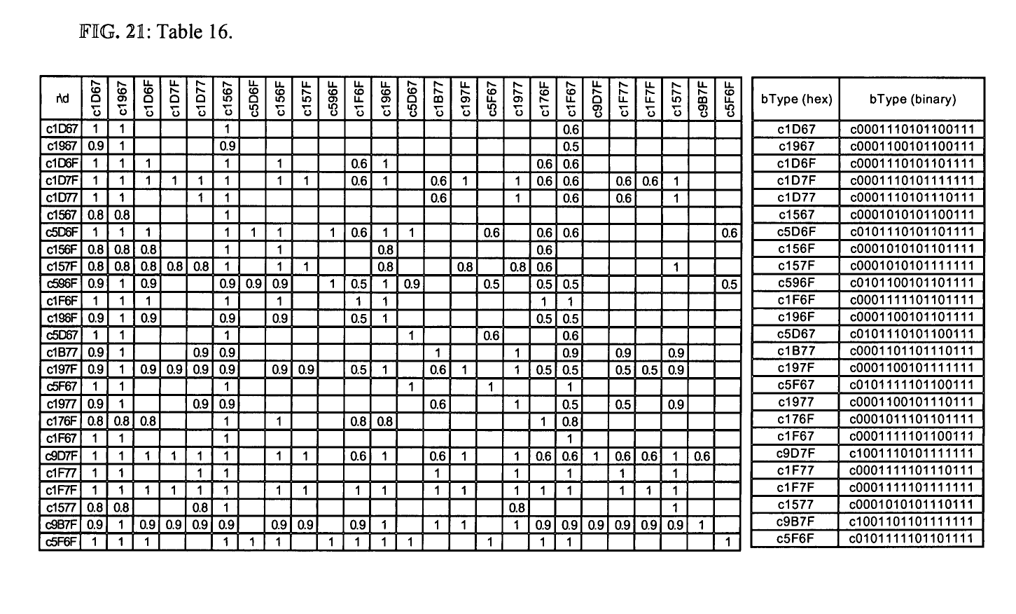

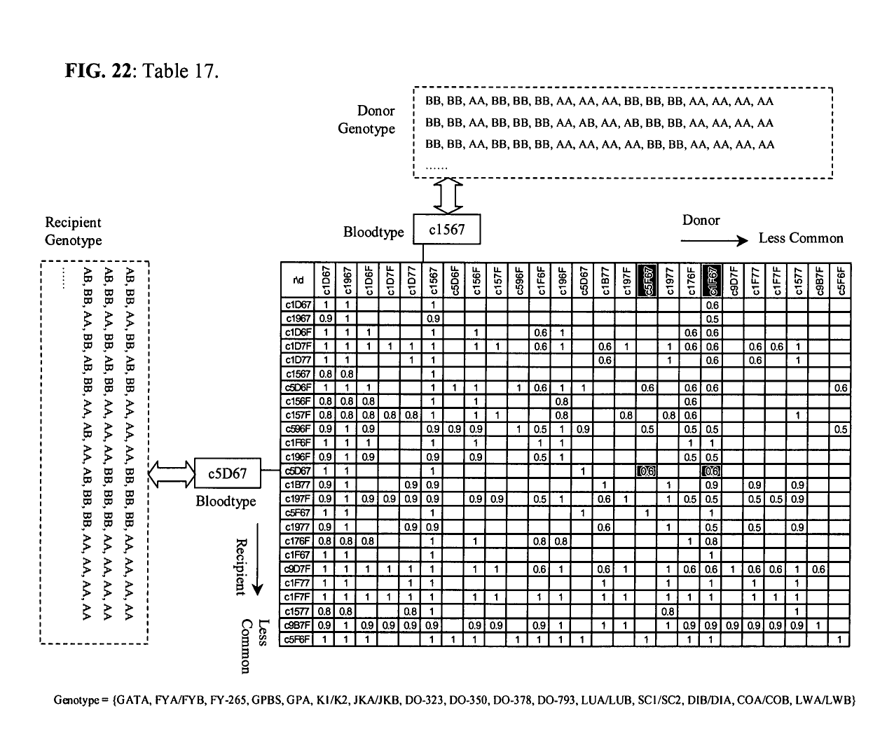

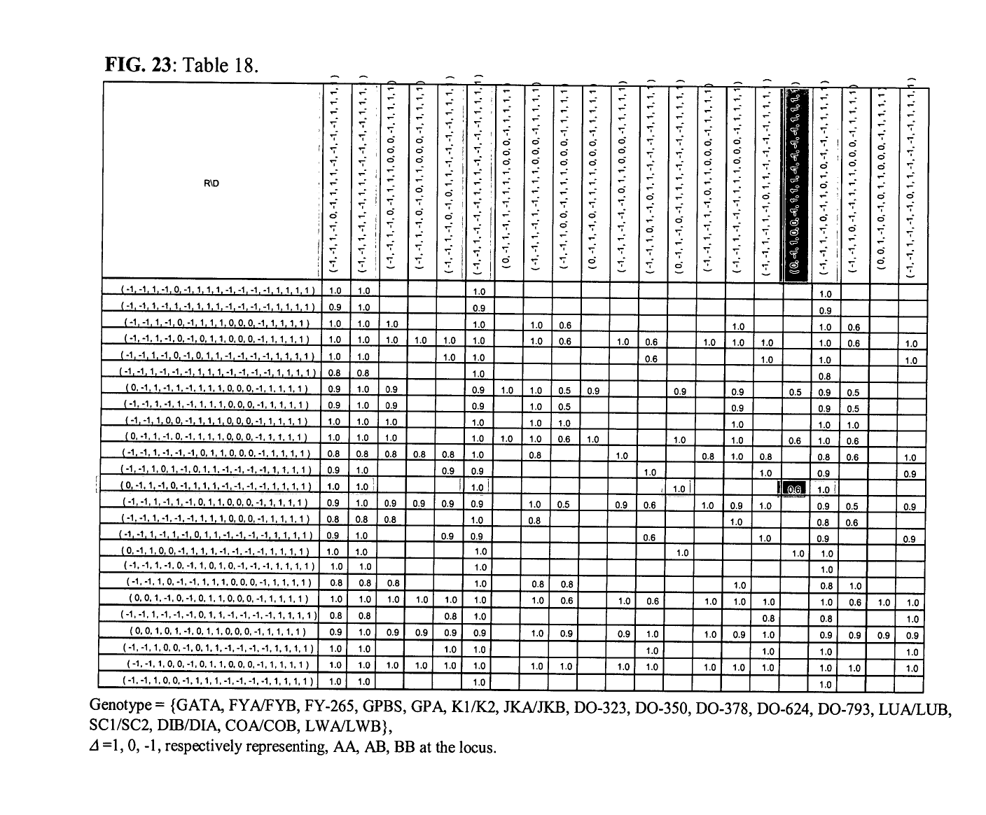

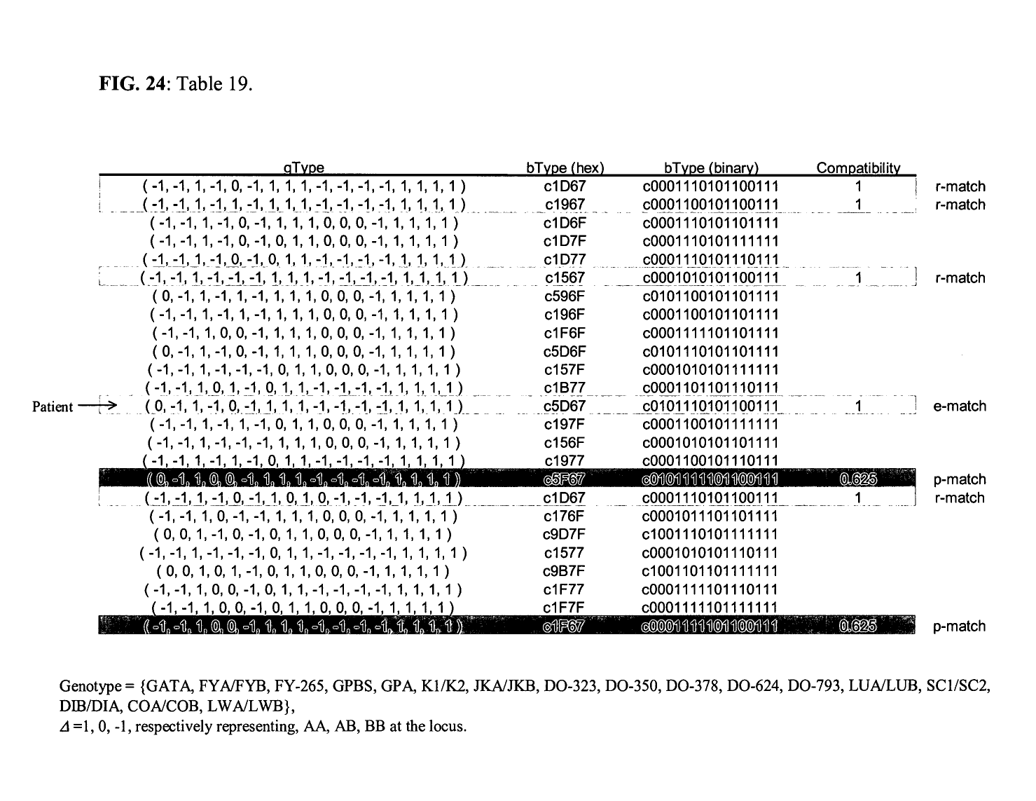

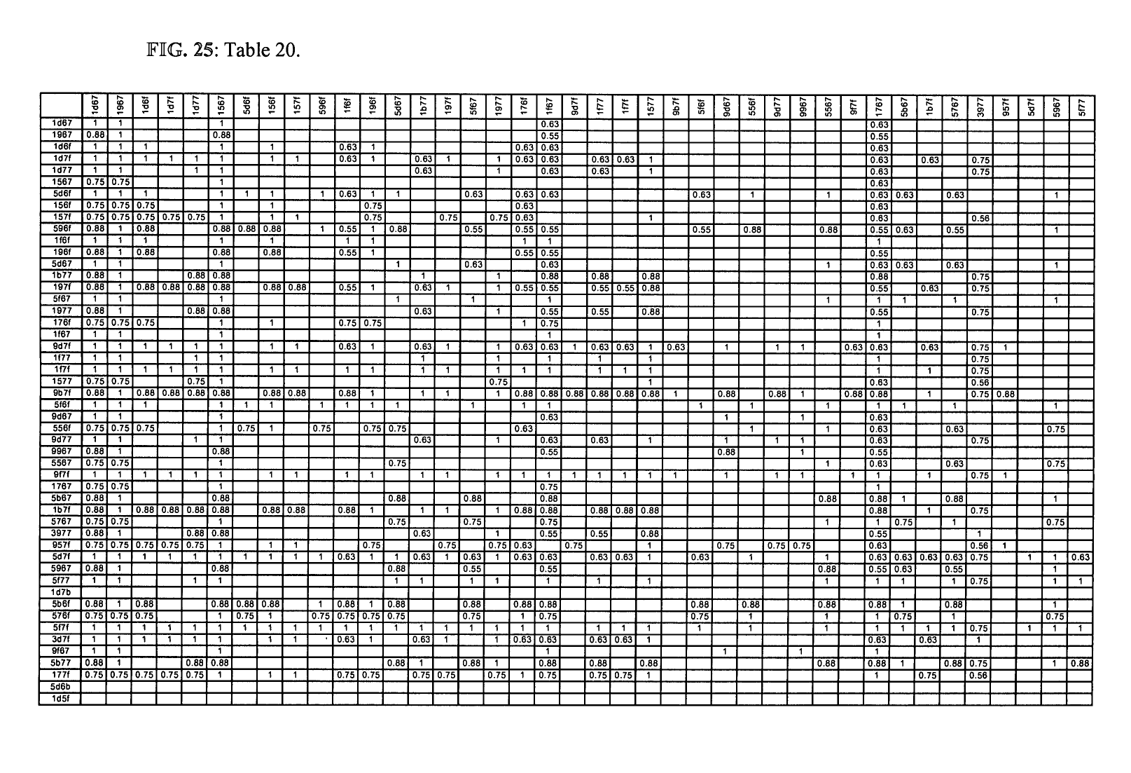

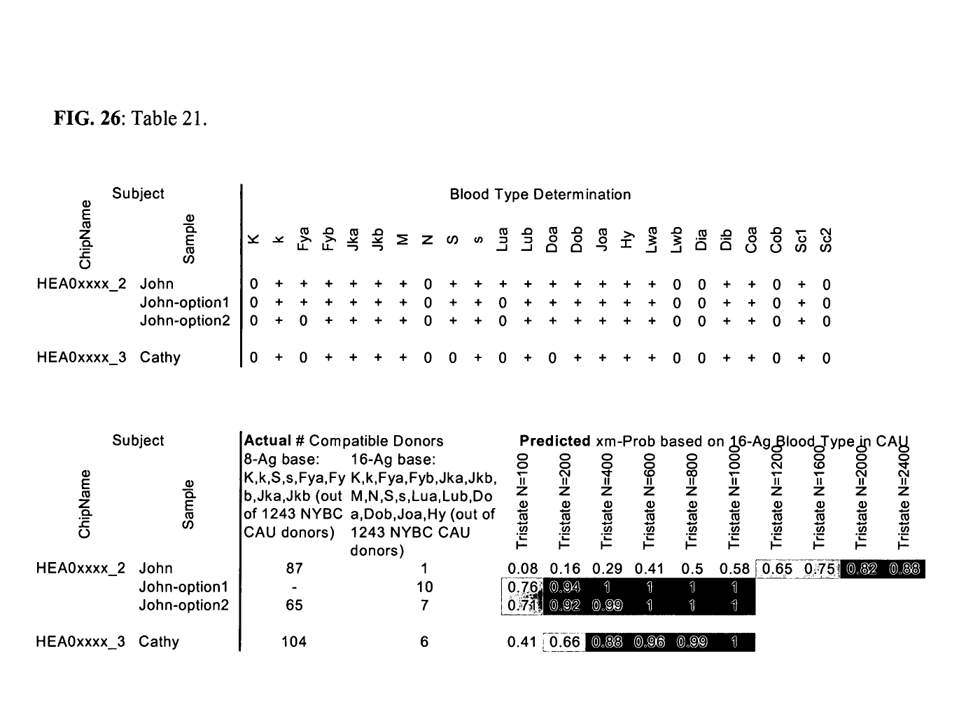

Blood Type Representation—Following phenotype mapping, each blood sample is then assigned a blood-type code, preferably a 16-bit string in this case. The antigen bits are arranged in the following order: Fy^{a}, Fy^{b}, Lu^{a}, Lu^{b}, M, N, S, s, K, k, Jk^{a}, Jk^{b}, Do^{a}, Do^{b}, Hy, Jo(a). The 20 most common blood types and their respective frequencies, as derived by genotype-to-phenotype and then phenotype-to-blood-type mapping, are listed in Table 13. To check the accuracy of the derived blood types is to compare the phenotype frequencies derived by the current method with those previously established by direct phenotyping using serological methods (Reid & Lomas-Francis, supra): as evident in Table 14, agreement is good, especially in view of the small cohort. Another way of validation is to compare the haplotype-derived frequencies with the frequencies derived by multiplying reported phenotype frequencies, assuming combination by pure chance. FIG. 5, in a bar chart representation, extends the comparison to all 53 blood types encountered; and, FIG. 6 displays the correlation between the two frequency sets, further supporting the validity of the genotype-derived blood types; the remaining discrepancies between the two sets, aside from the statistical fluctuations reflecting the small size of the cohort, may indicate a statistical correlation among some of the alleles in the selected panel.

Due to very limited sample size in this example, the identified haplotypes and frequencies may not be the most representative in African Americans. As a matter of fact, we derived a slightly different set of combinations and frequencies in a later large-scale study that involves over 2000 donors in New York region. Subsequently, genotype-to-phenotype mapping was subject to some minor changes. Tables and examples as disclosed herein are aimed at illustrating the principles of the current invention.

### EXAMPLE 6

### Cross-Matching in African American Population

Following the analysis in Example 5, a compatibility matrix was constructed by evaluating compatibility scores among the most frequent predicted blood types. Table 15 shows such a matrix for the 25 most common blood types derived from genotypes for African Americans after temporarily filtering out partially compatible blood types. The “1”'s along the diagonal indicate self-compatible blood types, representing compatible cross-match(es) in accordance with the Exact Cross-Matching Rule. As discussed, each blood type may correspond to multiple genotypes, as discussed in connection with Tables 3-5. The off-diagonal “1”'s represent compatible cross-match(es) in accordance with a Relaxed Cross-Matching Rule.

For example, again, take a blood type identified by the hexadecimal code c5D67 or the binary code c01011101011100111, that is (Fy^{a}−, Fy^{b}+, Lu^{a}−, Lu^{b}+, M+, N+, S−, s+, K−, k+, Jk^{a}+, Jk^{b}−, Do^{a}−, Do^{b}+, Hy+, Jo(a)+), or a combination of phenotypes, (Fy(a−^{b}+), Lu(a−b+), M+N+S−s+, K−k+, Jk(a+b−), Do(a−b+)). The compatibility matrix identifies three compatible codes, i.e., c1D67, c1967, and c1567, which respectively correspond to blood types,
(Fy^{a}−, Fy^{b}−, Lu^{a}−, Lu^{b}+, M+, N+, S−, s+, K−, k+, Jk^{a}+, Jk^{b}−, Do^{a}−, Do^{b}+, Hy+, Jo(a)+), (Fy^{a}−, Fy^{b}−, Lu^{a}, Lu^{b}+, M+, N−, S−, s+, K−, k+, Jk^{a}+, Jk^{b}−, Do^{a}−, Do^{b}+, Hy+, Jo(a)+) (Fy^{a}−, Fy^{b}−, Lu^{a}−, Lu^{b}+, M−, N+, S−, s+, K−, k+, Jk^{a}+, Jk^{b}−, Do^{a}−, Do^{b}+, Hy+, Jo(a)+),
   each characterized by the absence of one antigen, Fy^{b}, the absence of the two antigens, Fy^{b} and N, and the absence of the two antigens, Fy^{b} and M, respectively. As indicated by adding up all the frequencies of the compatible blood types, application of the Relaxed Cross-Matching Rule increases the chance of finding compatible donors to 22% for a blood type with a frequency of only 1.5%, even when just the 25 most frequent donor blood types are considered.

Partial Compatibility—A partial compatibility matrix also was constructed using mismatch scores, ranging from 0 to 1, for the antigens of interest in the order of decreasing severity level, as shown in Table 1. Table 16 shows the matrix for the 25 most common blood types in the African American population, setting to “0” (or simply leaving blank) all elements with compatibility scores below 0.5. Note that all elements of value “1” match those in Table 11; however, several fields left “blank” in the matrix of Table 11 now show finite scores corresponding to partially compatible donor blood types with compatibility scores greater than 0.5. Again, we take blood code c5D67. In Example 5, c5D67 identifies three compatible codes, i.e., c1D67, c1967, and c1567. In this example, in addition to those three fully compatible codes, two more codes, i.e., 5F67 and 1F67, are found partially compatible, which respectively correspond to blood types,
(Fy^{a}−, Fy^{b}+, Lu^{a}−, Lu^{b}+, M+, N+, S+, s+, K−, k+, Jk^{a}+, Jk^{b}−, Do^{a}−, Do^{b}+, Hy+, Jo(a)+), (Fy^{a}−, Fy^{b}−, Lu^{a}−, Lu^{b}+, M+, N+, S+, s+, K−, k+, Jk^{a}+, Jk^{b}−, Do^{a}−, Do^{b}+, Hy+, Jo(a)+);
   Compared to recipient code c5D67, donor code c5F67 comprises the moderately offending antigen, S, and the partial compatibility score, 0.625, suggests a moderate acceptability. The code c1F67 comprises the null phenotype Fy(a−b−) for Duffy which is compatible under the Relaxed Cross-Matching Rule, but also comprises the moderately offending antigen, S, rendering its overall partial compatibility to recipient code c5D67 comparable to that of c5F67.

### EXAMPLE 7

### Rapid Search of Compatible Donors in African American Population

Suppose a recipient with blood type code c5D67 places a request for compatible donors in an African American donor pool. A priority list of potentially compatible donor blood types is first constructed by “look-up” in an established compatibility matrix such as Table 14: the row assigned to c5D67, shows six potentially compatible blood types. Next, the search list is constructed to contain a top-priority blood code—c5D67—identical to that of the recipient, and a medium-priority section containing r-matches sorted by their occurrence frequencies—c1D67, c1967, c1567, and c5D67, and a third section of low-priority blood types (the p-matches), containing c5F67 and c1F67—the partially compatible blood types.

### EXAMPLE 8

### Genotype Cross-Matching and Search

Table 17 shows genotype compatibility matrix for the African American population derived from the blood type compatibility matrix in Table 16 and discussed in Examples 7 and 8. In the new matrix, rows and columns are assigned to genotypes, and the matrix element at the intersection of a specific row (recipient genotype) and column (donor genotype) contains the compatibility score of for the corresponding blood types. Table 18 shows a genotype compatibility matrix for the 50 most common 16-antigen minor-group genotypes in an African American population. For a patient, with given genotype (0, −1, 1, −1, 0, −1, 1, 1, 1, −1, −1, −1, −1, 1, 1, 1, 1), compatible donor genotypes among those 50 choices, as shown in Table 19, include: one e-Match, namely the identical code, as well as:

four r-Matches, namely:

(−1, −1, 1, −1, 0, −1, 1, 1, 1, −1, −1, −1, −1, 1, 1, 1, 1);
(−1, −1, 1, −1, 1, −1, 1, 1, 1, −1, −1, −1, −1, 1, 1, 1, 1);
(−1, −1, 1, −1, −1, −1, 1, 1, 1, −1, −1, −1, −1, 1, 1, 1, 1); and
(−1, −1, 1, −1, 0, −1, 1, 0, 1, 0, −1, −1, −1, 1, 1, 1, 1);
   and two p-matches, namely:
(0, −1, 1, 0, 0, −1, 1, 1, 1, −1, −1, −1, −1, 1, 1, 1, 1); and
(−1, −1, 1, 0, 0, −1, 1, 1, 1, −1, −1, −1, −1, 1, 1, 1, 1)

### EXAMPLE 9

### Finding Compatible Blood for Two Caucasian Individuals in an Actual Caucasian Donor Pool in the New York Region

A pool of more than 2300 potential donors of diverse ethnic background were analyzed using the BeadChip™ platform. Phenotypes derived from DNA analysis were concordant with 4,510 of the 4,534 pairs of partial antigen determinations made by hemagglutination for the MNS, Lutheran, Kell, Duffy, Kidd, Dombrock, and Colton blood group systems. Of the 24 discordant results, 16 were resolved by sequencing and RFLP analysis in favor of the BeadChip™ results. The other 8 discordant results were shown to be due to silencing of GYPB—the relevant SNPs were subsequently added to a later version of HEA BeadChip™ panel (see Hashmi et al., Determination of 24 Minor Red Blood Cell Antigens for More Than 2000 Blood Donors by High-Throughput DNA Analysis, Manuscript ID Trans-2006-0329, R 1, Transfusion, 2006.)

Two Caucasian individuals volunteered to have their blood antigens typed. To keep their anonymity, we rename them “John” and “Cathy”. DNA typing over the set {GATA, FY, FY-265, GPA, GPB, K, Jk, DO-323, DO-350, DO-378, DO-624, DO-793, Lu, SC, DI, CO, LW} shows John has type (K−, k+, Fy^{a}+, Fy^{b}+, M+, N−, S+, s+, Lu^{a}+, Lu^{b}+, Do^{a}+, Do^{b}+, Jo(a)+, Hy+, Lw^{a}+, Lw^{b}−, Di^{a}−, Di^{b}+, Co^{a}+, Co^{b}−, Sc1+, Sc2−), or binary code (c0111101111111110011010), and Cathy has type (K−, k+, Fy^{a}−, Fy^{b}+, M+, N−, S−, s+, Lu^{a}−, Lu^{b}+, Do^{a}−, Do^{b}+, Jo(a)+, Hy+, Lw^{a}+, Lw^{b}−, Di^{a}−, Di^{b}+, Co^{a}+, Co^{b}−, Sc1+, Sc2−), or binary code (c0101100101011110011010), John's blood is a rare combination of antigens due to a rare positive Lu^{a} antigen, whose corresponding LUA allele observed in only 3% Caucasians. If matching is based on 8-antigens: K, k, S, s, Fy^{a}, Fy^{b}, Jk^{a}, Jk^{b}, John can find 87 exact matches out of a subset of 1243 Caucasian individuals in the donor pool; however, if 8 additional antigens are included—M, N, Lu^{a}, Lu^{b}, Do^{a}, Do^{b}, Joa, and Hy—John can find only one exact match in the subset. The estimated frequency of John's extended type, following method disclosed herein, is a mere 0.09% in the CAU cohort, consistent with the observation that only one match was found in the CAU cohort. On the other hand, if relaxed matching rule is followed, we immediately find at least two compatible blood types that are expected to occur with high frequencies in the Caucasians, i.e., option 1 (K−, k+, Fy^{a}+, Fy^{b}+, M+, N−, S+, s+, Lu^{a}−, Lu^{b}+, Do^{a}+, Do^{b}+, Jo(a)+, Hy+, Lw^{a}+, Lw^{b}−, Di^{a}−, Di^{b}+, Co^{a}+, Co^{b}−, Sc1+, Sc2−, f=1.43%), or binary code (c011101101111110011010), with Lu^{a} being negative, and option 2 (K−, k+, Fy^{a}−, Fy^{b}+, M+, N−, S+, s+, Lu^{a}−, Lu^{b}+, Do^{a}+, Do^{b}+, Jo(a)+, Hy+, Lw^{a}+, Lw^{b}−, Di^{a}−, Di^{b}+, Co^{a}+, Co^{b}−, Sc1+, Sc2−, f=1.24%), or binary code (c0101101101111110011010), with Lu^{a} and Fy both being negative.

Table 21 shows cross-matching probabilities predicted, by using the expression disclosed in a pending patent application (Zhang et al, “A Transfusion Registry and Exchange Network,” U.S. Ser. No. 11/412,667, Apr. 27, 2006, incorporated by reference), of finding at least one cross-matched compatible donor in different size of randomly recruited donor set. For example, the probability of finding either blood type in a group of 200 randomly selected Caucasian donors is greater than 90%. A search within the Caucasian cohort (N=1243) produced 10 and 7 matched compatible donors, respectively, for blood type option 1 and option 2, consistent with the prediction.

Cathy's type is more common than John's which has a frequency of 0.53%. Predicted cross-matching probabilities in 200 and 400 random Caucasian donors are, respectively, 66% and 88%. Search of compatible donors in the Caucasian subset produced six 16-antigen exact matches, again consistent with the prediction, within the error of sampling fluctuations.

What is claimed is:

1. A method of identifying blood product donors compatible with a particular recipient comprising:
representing candidate donor and recipient minor blood types as bit strings, where one value of a bit represents that a particular blood type antigen is present and another value represents that said antigen is not present, and where the bit strings comprise blocks of at least two bits representing the antigen configurations of specific phenotypes; and
matching the candidate donor and recipient bit strings by forming a Boolean expression wherein the expression yields a first value in the event of a match, indicating compatibility, and second value in the event of a mismatch, indicating incompatibility, and the results of the Boolean expression are recorded.

2. The method of claim 1 wherein, in the recorded results, compatibility is indicated by a value of TRUE (“1”) of the string-matching expression, and incompatibility is indicated by a value of FALSE (“0”).

3. The method of claim 2 wherein the Boolean expression represents, respectively, compatibility or incompatibility under either a cross-matching criterion where the candidate donor and the recipient have the same antigens or a cross-matching criterion where the candidate donor does not have any antigens the recipient does not have.

4. The method of claim 3 wherein the cross-matching criterion requiring that the candidate not have any antigens the recipient does not have is represented by a Boolean expression involving donor code and recipient code, in which the recipient code, [βᵣ], serves as a mask to zero all the bits of the donor blood type code [β_{d}] for which the corresponding bit in [βᵣ] is 1.

5. The method of claim 1 wherein, when comparing the blood type of a recipient to the blood types of multiple candidate donors of potentially compatible blood type, the results are recorded in the form of a compatibility vector.

6. The method of claim 1 wherein, when comparing the blood types of multiple recipients to the blood types of multiple candidate donors of potentially compatible blood type, the results are recorded in the form of a compatibility matrix.

7. The method of claim 1 wherein the method is applied to identify prospective matches in a registry of typed donors.

8. The method of claim 7 wherein the identification is performed using a real-time search algorithm.

9. The method of claim 1 wherein the representation of candidate donor and recipient strings is in binary, octal or hexadecimal form.

10. The method of claim 1 wherein the bit string representing a recipient blood type is augmented to include additional antigens to which the recipient has formed antibodies.

11. A method of identifying blood product donors compatible with a particular recipient comprising:
representing candidate donor and recipient minor blood types as bit strings, where a value of a bit represents that a particular minor blood type antigen is expressed and another value represents that said antigen is not expressed, or must not be expressed;
matching the candidate donor and recipient strings;
identifying mismatched bits and assigning to each a mismatch score reflecting the clinical significance of the mismatch; and
multiplying the scores to determine a partial compatibility score and thereby assigning a risk to transfusing the partially compatible blood product by comparing the partial compatibility score with a threshold indicating the limits of acceptable risk.

12. The method of claim 11 wherein strings are compared by application of a Boolean operation to the candidate donor and the recipient string, forming a Boolean expression indicating incompatibility or compatibility.

13. The method of claim 12 wherein incompatibility is established by the Boolean expression producing a value of FALSE (“0”) and compatibility is established by the Boolean expression producing a value of TRUE (“1”).

14. The method of claim 11 wherein the values of the bits are encoded with a binary, octal or hexadecimal code.

15. The method of claim 11 wherein the mismatch scores are between 1 and 0 and mismatch scores of greater clinical significance are indicated by scores closer to 0.

16. The method of claim 11 wherein the cross-matching criterion applied to each bit is either: (i) that the donor and recipient strings are identical at that bit; or (ii) that the donor and recipient strings are identical at that bit and the donor does not express an antigen at positions (as indicated at corresponding bits) where the recipient does express an antigen.

17. The method of claim 11 wherein the bit string representing a recipient blood type is augmented to include additional antigens to which the recipient has formed antibodies.

18. The method of claim 11 wherein, in the event of incompatibility, mismatched bits are identified.

19. A method of representing (and/or a representation of) the pair-wise compatibilities between a selected set of minor blood groups in the form of a matrix, wherein blood groups are in the form of bit strings wherein one value of a bit represents that the corresponding particular minor blood type antigen is present and another value represents that said antigen is not present, the method comprising:
placing a value of “0” into fields corresponding to pairs of incompatible blood types; and
placing a positive value into fields corresponding to pairs of at least partially compatible blood types.

20. The method of claim 19 wherein the positive value is a value of “1” when pairs of blood types are compatible under an Exact Cross-Matching Rule or under a Relaxed Cross-Matching Rule and a value in the range (0,1) when pairs of blood types are partially compatible.

21. The representation of claim 19 wherein the bit strings are represented in binary, octal or hexadecimal form.

22. A method for determining whether or not to administer a transfusion, on the basis the genotypes of a prospective donor and a recipient, comprising, in any order except as otherwise provided below:
(i) determining genotypes of prospective donors and recipient using, for each of a designated set of variable sites within genes controlling the expression of selected potentially immunogenic antigens, a pair of degenerate probes permitting, at each such site, an assignment as homozygous normal, homozygous variant or heterozygous, the set of such recorded assignments constituting the genotype;
(ii) decomposing said donor and recipient genotypes into combinations of donor and recipient haplotypes, the sites in a haplotype designated with either a value indicating normal or a value indicating variant, the combination of a pair of haplotypes yielding the genotype;
(iii) correlating said haplotypes with phenotypes by application of rules of inheritance for the selected antigens;
(iv) in the event of ambiguity in haplotype assignment, indicated by two or more haplotype combinations being consistent with the genotype, and at least two of these combinations mapping to different phenotypes, assigning a maximal risk, determined by identifying the maximally incompatible phenotypes among the different possible donor and recipient phenotype combinations determined from correlating phenotypes with haplotype combinations for donor and recipient, wherein incompatibility is based on the degree of clinical significance of the mismatched antigens in the donor and recipient phenotypes, and representing the degree of clinical significance of said donor/recipient mismatches by computing a partial compatibility score representing the cumulative effect of all mismatches in a particular phenotype of each of donor and recipient;
(v) in the event the maximal risk represents a risk greater than a risk threshold, reducing the ambiguity by selecting as the haplotypes those estimated to occur most frequently in the population of recipients and donors and re-computing the partial compatibility score(s) represented by the phenotypes corresponding to said selected haplotypes;
(vi) in the event of the maximal risk represents a risk greater than a risk threshold after step (v) is performed, resolving the gametic phase to determine the actual haplotype and eliminate ambiguity in the phenotype mapping; and
(vii) determining compatibility by matching donor and recipient phenotypes and determining whether there is an exact match at all sites or, in the event of a mismatch at certain sites, determining whether it is a mismatch which is tolerated under the matching rules in effect, or because of the partial compatibility score.

23. The method of claim 22 wherein the gametic phase is resolved using probe pairs, wherein the probes are designed to resolve the ambiguity in haplotype combinations by resolving the gametic phase.

24. The method of claim 22 wherein phasing is used to determine sites which do not themselves code antigens but which control the expression (or the silencing of the expression) of antigens.

25. The method of claim 22 wherein a prospective donor is classified as compatible to a given recipient if the prospective donor and recipient express the same antigens, or the donor does not express any antigens which the recipient does not express, or, in the event that these conditions are not met, the score of maximal risk is below a threshold.

26. The method of claim 22 wherein the ambiguity in phenotype assignment is reduced by selecting as the likely haplotypes those estimated to occur most frequently in the population of recipients and donors.

27. The method of claim 22 wherein haplotypes estimated to occur most frequently are determined by gene counting or by application of an Expectation Maximization algorithm.

28. The method of claim 22 further including the step of ranking degenerate haplotype combinations by estimated frequency of occurrence in the populations, respectively, of prospective donor and recipient, and removing from consideration haplotypes with an estimated frequency of occurrence below a threshold.

29. The method of claim 22 wherein the maximal risk is assigned by determining the product of the assigned value of clinical risk at each site where there is a phenotype mismatch between prospective donor and recipient.

30. The method of claim 22 wherein the pattern of compatibility between pairs of phenotypes in recipients and prospective donors is recorded in a compatibility matrix.

31. The method of claim 22 further including the step of determining the likelihood that certain haplotypes which result from the decomposition occur, based on known frequencies of occurrence in a population.

32. The method of claim 31 wherein the likelihood determined is used in conjunction with the clinical significance of a mismatch to assess risk of incompatibility.

33. A method for the determination of the degree of compatibility of a prospective blood product donor to a recipient, on the basis of the transfusion antigen genotypes of said donor and said recipient, said transfusion antigen genotypes comprising the combination of alleles at designated variable loci affecting the expression of particular transfusion antigens defining a phenotype, comprising:
mapping the transfusion antigen genotype to corresponding phenotypes by decomposing the genotype into haplotype combinations and determining the antigen expression state under rules of inheritance;
in the event of ambiguity in mapping, indicated by two or more haplotype combinations giving a genotype but producing different antigen expression states, reducing or resolving the ambiguity; and
determining the compatibility of the transfusion phenotypes (or blood types) of prospective donor and recipient.

34. The method of claim 33 wherein ambiguity is reduced or resolved by eliminating haplotype combinations having an estimated frequency of occurrence below a threshold.

35. The method of claim 33 or 34 wherein a score of the risk associated with ambiguity in mapping is obtained by identifying such positions within the bit strings representing different mapped phenotypes at which at least one bit string differs from the others, computing the product of mismatch scores reflecting the degree of clinical significance of potentially mismatched antigens at such identified positions in the donor and recipient phenotypes, the product representing the cumulative effect of all mismatches between the different mapped phenotypes.

36. The method of claim 35 wherein in the event the product represents a risk greater than a risk threshold, reducing the ambiguity by selecting as the haplotypes those estimated to occur most frequently in the population of recipients and donors and re-computing the partial compatibility score(s) represented by the phenotypes corresponding to said selected haplotypes.

37. The method of claim 35 wherein in the event the product represents a risk greater than a risk threshold, the ambiguity is resolved by gametic phasing.

38. The method of claim 37 wherein the gametic phase is resolved using probe pairs, wherein the probes are designed to resolve the ambiguity in haplotype combinations by resolving the gametic phase.

39. The method of claim 37 wherein the haplotypes are selected based on visual inspection of existing data, or gene counting, preferably by application of an Expectation Maximization algorithm.

40. The method of claim 33 wherein the donor and recipient phenotypes (and their corresponding blood groups) mapped and decomposed to haplotypes are as follows:

| Blood Group | Antigens |
|---|---|
| Colton | Co^{a}/Co^{b} |
| Diego | Di^{b}/Di^{a} |
| Duffy | Fy^{a}/Fy^{b} |
| | Fy^{x} [Fy(b+^{w})] |
| | Fy(a−b−) |
| Dombrock | Do^{a}/Do^{b} |
| | Hy+/Hy− |
| | Jo(a+)/Jo(a−) |
| Kidd | Jk^{a}/Jk^{b} |
| Kell | K/k |
| Landsteiner-Wiener | LW^{a}/LW^{b} |
| Lutheran | Lu^{a}/Lu^{b} |
| MNS | M/N |
| | S/s |
| Scianna | Sc1/Sc2 |
| Rh | C/c |
| Rh | E/e |

41. The method of claim 33 further including the step of determining the likelihood that certain haplotypes which result from the decomposition occur, based on known frequencies of occurrence in a population.

42. The method of claim 41 wherein the likelihood determined is used in conjunction with the clinical significance of a mismatch to assess risk of incompatibility.

43. A method of establishing the compatibility of first and second genotypes, each genotype comprising designated variable loci controlling the expression of minor blood group antigens, wherein the genotype, at each locus, is determined as normal, variant or heterozygous by targeting each locus with a pair of probes, a positive result produced by one probe in each pair indicating a normal, and a positive result produced by the other probe in the pair indicating a variant, comprising:
mapping first and second genotypes to first and second sets of antigens defining phenotypes;
establishing the compatibility of first and second phenotypes under a preset cross-matching criterion;
wherein the compatibility of said first and second phenotypes determines the compatibility of first and second genotypes under said cross-matching criterion.

44. The method of claim 43 wherein the designated variable loci comprise at least the group in the table below:

| Blood Group | Variable Site |
|---|---|
| Colton | CO134C>T |
| Diego | DI2561T>C |
| Duffy | FY-33T>C |
| | FY125G>A |
| | 265C>T |
| Dombrock | DO-323G>T |
| | DO-350C>T |
| | DO-793A>G |
| Kidd | JK838G>A |
| Kell | KEL698T>C |
| Landsteiner-Wiener | LW308A>G |
| Lutheran | LU230A>G |
| MNS | GYPA 59C>T |
| | GYPB 143T>C |
| Scianna | SC169G>A |
| Rh-CE | P103S |
| | A226P |

45. The method of claim 43 wherein under said mapping, a genotype uniquely maps to one phenotype and therefore identity of first and second genotypes unambiguously indicates compatibility.

46. The method of claim 45 wherein the minor blood group genotypes are LU, JK, K, GPA or GPB.

47. The method of claim 43 wherein first and second genotypes are those of a candidate blood product donor and a recipient, respectively, and the cross-matching criterion is either an exact cross-matching criterion, wherein candidate donor and recipient have the same antigens, or a cross-matching criterion wherein the candidate donor does not have any antigens the recipient does not.
